SEQUENCE LISTING (86 sequences)

SEQ ID NO: 1 - protein, 8 aa, Saccharomyces cerevisiae
GYSFTSYW

SEQ ID NO: 2 - protein, 8 aa, Saccharomyces cerevisiae
IYPGDSDT

SEQ ID NO: 3 - protein, 17 aa, Saccharomyces cerevisiae
CARLGYYDSSGYYSEDY

SEQ ID NO: 4 - protein, 7 aa, Saccharomyces cerevisiae
SGRSGFA

SEQ ID NO: 5 - protein, 7 aa, Saccharomyces cerevisiae
VNSDGSH

SEQ ID NO: 6 - protein, 9 aa, Saccharomyces cerevisiae
QTWGTGIWV

SEQ ID NO: 7 - protein, 131 aa, Saccharomyces cerevisiae
QVQLVQSGAEVKKPGEPLKISCKGSGYSFTSYWIGWVRQMPGKGLEWMGIIYPGDSDTRYSPSFQGQVTISADKSISTAYLQWSSLKASDTAMYYCARLGYYDSSGYYSEDYWGQGTLVTVSSGSASAPTL

SEQ ID NO: 8 - protein, 127 aa, Saccharomyces cerevisiae
QPVLTQSSSASASLGASVKLTCTLSSGRSGFAIAWHQQHPEKGPRYLMNVNSDGSHTKGDGIPDRFSGSSSEAERYLTISSLQFDDEVDYYCQTWGTGIWVFGGGTKLTVLRQPKAAPSVTLFPAVL

SEQ ID NO: 9 - DNA, 15 bp, Saccharomyces cerevisiae
aaaattggtattata

SEQ ID NO: 10 - DNA, 15 bp, Saccharomyces cerevisiae
caatacgaactctta

SEQ ID NO: 11 - DNA, 21 bp, Artificial Sequence
  Primer sequence
attctgaggttgctgctttgg

SEQ ID NO: 12 - DNA, 22 bp, Artificial Sequence
  Primer sequence
tgtcttggtctaccgacgatag

SEQ ID NO: 13 - DNA, 20 bp, Artificial Sequence
  Primer sequence
tcgccgcaatgtctactacc

SEQ ID NO: 14 - DNA, 20 bp, Artificial Sequence
  Primer sequence
gcccaattgtgcttgctgaa

SEQ ID NO: 15 - DNA, 19 bp, Artificial Sequence
  Primer sequence
tgcctgcttgccgaatatc

SEQ ID NO: 16 - DNA, 20 bp, Artificial Sequence
  Primer sequence
atatcacgggtagccaacgc

SEQ ID NO: 17 - DNA, 23 bp, Artificial Sequence
  Primer sequence
caggtbcagctggtgcagtctgg

SEQ ID NO: 18 - DNA, 23 bp, Artificial Sequence
  Primer sequence
carrtscagctggtrcartctgg

SEQ ID NO: 19 - DNA, 23 bp, Artificial Sequence
  Primer sequence
cagrtcaccttgaaggagtctgg

SEQ ID NO: 20 - DNA, 23 bp, Artificial Sequence
  Primer sequence
sargtgcagctggtggagtctgg

SEQ ID NO: 21 - DNA, 23 bp, Artificial Sequence
  Primer sequence
gaggtgcagctgktggagwcysg

SEQ ID NO: 22 - DNA, 23 bp, Artificial Sequence
  Primer sequence
caggtgcarctgcaggagtcggg

SEQ ID NO: 23 - DNA, 23 bp, Artificial Sequence
  Primer sequence
cagstgcagctrcagsagtssgg

SEQ ID NO: 24 - DNA, 23 bp, Artificial Sequence
  Primer sequence
gargtgcagctggtgcagtctgg

SEQ ID NO: 25 - DNA, 23 bp, Artificial Sequence
  Primer sequence
caggtacagctgcagcagtcagg

SEQ ID NO: 26 - DNA, 21 bp, Artificial Sequence
  Primer sequence
aagggttggggcggatgcact

SEQ ID NO: 27 - DNA, 23 bp, Artificial Sequence
  Primer sequence
gacatccagatgacccagtctcc

SEQ ID NO: 28 - DNA, 23 bp, Artificial Sequence
  Primer sequence
gmcatccrgwtgacccagtctcc

SEQ ID NO: 29 - DNA, 23 bp, Artificial Sequence
  Primer sequence
gatrttgtgatgacycagwctcc

SEQ ID NO: 30 - DNA, 23 bp, Artificial Sequence
  Primer sequence
gaaatwgtgwtgacrcagtctcc

SEQ ID NO: 31 - DNA, 23 bp, Artificial Sequence
  Primer sequence
gacatcgtgatgacccagtctcc

SEQ ID NO: 32 - DNA, 23 bp, Artificial Sequence
  Primer sequence
gaaacgacactcacgcagtctcc

SEQ ID NO: 33 - DNA, 23 bp, Artificial Sequence
  Primer sequence
gawrttgtgmtgacwcagtctcc

SEQ ID NO: 34 - DNA, 24 bp, Artificial Sequence
  Primer sequence
acactctcccctgttgaagctctt

SEQ ID NO: 35 - DNA, 23 bp, Artificial Sequence
  Primer sequence
cagtctgtgctgactcagccacc

SEQ ID NO: 36 - DNA, 23 bp, Artificial Sequence
  Primer sequence
cagtctgtgbtgacgcagccgcc

SEQ ID NO: 37 - DNA, 21 bp, Artificial Sequence
  Primer sequence
cartctgccctgactcagcct

SEQ ID NO: 38 - DNA, 23 bp, Artificial Sequence
  Primer sequence
tcctatgwgctgacwcagccacc

SEQ ID NO: 39 - DNA, 23 bp, Artificial Sequence
  Primer sequence
tcttctgagctgactcaggaccc

SEQ ID NO: 40 - DNA, 23 bp, Artificial Sequence
  Primer sequence
cacgttatactgactcaaccgcc

SEQ ID NO: 41 - DNA, 23 bp, Artificial Sequence
  Primer sequence
cagcytgtgctgactcaatcryc

SEQ ID NO: 42 - DNA, 21 bp, Artificial Sequence
  Primer sequence
ctgcctgtgctgactcagccc

SEQ ID NO: 43 - DNA, 20 bp, Artificial Sequence
  Primer sequence
cagsctgtgctgactcagcc

SEQ ID NO: 44 - DNA, 23 bp, Artificial Sequence
  Primer sequence
aattttatgctgactcagcccca

SEQ ID NO: 45 - DNA, 23 bp, Artificial Sequence
  Primer sequence
cagrctgtggtgacycaggagcc

SEQ ID NO: 46 - DNA, 23 bp, Artificial Sequence
  Primer sequence
cwgccwgkgctgactcagccmcc

SEQ ID NO: 47 - DNA, 23 bp, Artificial Sequence
  Primer sequence
tgaacattctgtaggggccactg

SEQ ID NO: 48 - DNA, 23 bp, Artificial Sequence
  Primer sequence
tgaacattccgtaggggcaactg

SEQ ID NO: 49 - DNA, 47 bp, Artificial Sequence
  Primer sequence
gcactaattaatgtggcctcaggacaggtbcagctggtgcagtctgg

SEQ ID NO: 50 - DNA, 47 bp, Artificial Sequence
  Primer sequence
gcactaattaatgtggcctcaggacarrtscagctggtrcartctgg

SEQ ID NO: 51 - DNA, 47 bp, Artificial Sequence
  Primer sequence
gcactaattaatgtggcctcaggacagrtcaccttgaaggagtctgg

SEQ ID NO: 52 - DNA, 47 bp, Artificial Sequence
  Primer sequence
gcactaattaatgtggcctcaggasargtgcagctggtggagtctgg

SEQ ID NO: 53 - DNA, 47 bp, Artificial Sequence
  Primer sequence
gcactaattaatgtggcctcaggagaggtgcagctgktggagwcysg

SEQ ID NO: 54 - DNA, 47 bp, Artificial Sequence
  Primer sequence
gcactaattaatgtggcctcaggacaggtgcarctgcaggagtcggg

SEQ ID NO: 55 - DNA, 47 bp, Artificial Sequence
  Primer sequence
gcactaattaatgtggcctcaggacagstgcagctrcagsagtssgg

SEQ ID NO: 56 - DNA, 47 bp, Artificial Sequence
  Primer sequence
gcactaattaatgtggcctcaggagargtgcagctggtgcagtctgg

SEQ ID NO: 57 - DNA, 47 bp, Artificial Sequence
  Primer sequence
gcactaattaatgtggcctcaggacaggtacagctgcagcagtcagg

SEQ ID NO: 58 - DNA, 42 bp, Artificial Sequence
  Primer sequence
ggaagatctagaggaaccaccaagggttggggcggatgcact

SEQ ID NO: 59 - DNA, 77 bp, Artificial Sequence
  Primer sequence
ggtggttcctctagatcttcctcctctggtggcggtggctcgggcggtggtggggacatccagatgacccagtctcc

SEQ ID NO: 60 - DNA, 77 bp, Artificial Sequence
  Primer sequence
ggtggttcctctagatcttcctcctctggtggcggtggctcgggcggtggtggggmcatccrgwtgacccagtctcc

SEQ ID NO: 61 - DNA, 77 bp, Artificial Sequence
  Primer sequence
ggtggttcctctagatcttcctcctctggtggcggtggctcgggcggtggtggggatrttgtgatgacycagwctcc

SEQ ID NO: 62 - DNA, 77 bp, Artificial Sequence
  Primer sequence
ggtggttcctctagatcttcctcctctggtggcggtggctcgggcggtggtggggaaatwgtgwtgacrcagtctcc

SEQ ID NO: 63 - DNA, 77 bp, Artificial Sequence
  Primer sequence
ggtggttcctctagatcttcctcctctggtggcggtggctcgggcggtggtggggacatcgtgatgacccagtctcc

SEQ ID NO: 64 - DNA, 77 bp, Artificial Sequence
  Primer sequence
ggtggttcctctagatcttcctcctctggtggcggtggctcgggcggtggtggggaaacgacactcacgcagtctcc

SEQ ID NO: 65 - DNA, 77 bp, Artificial Sequence
  Primer sequence
ggtggttcctctagatcttcctcctctggtggcggtggctcgggcggtggtggggawrttgtgmtgacwcagtctcc

SEQ ID NO: 66 - DNA, 48 bp, Artificial Sequence
  Primer sequence
cttgtcgtcatcgtctttgtagtcgaagacagatggtgcagccacagt

SEQ ID NO: 67 - DNA, 77 bp, Artificial Sequence
  Primer sequence
ggtggttcctctagatcttcctcctctggtggcggtggctcgggcggtggtgggcagtctgtgctgactcagccacc

SEQ ID NO: 68 - DNA, 77 bp, Artificial Sequence
  Primer sequence
ggtggttcctctagatcttcctcctctggtggcggtggctcgggcggtggtgggcagtctgtgbtgacgcagccgcc

SEQ ID NO: 69 - DNA, 75 bp, Artificial Sequence
  Primer sequence
ggtggttcctctagatcttcctcctctggtggcggtggctcgggcggtggtgggcartctgccctgactcagcct

SEQ ID NO: 70 - DNA, 77 bp, Artificial Sequence
  Primer sequence
ggtggttcctctagatcttcctcctctggtggcggtggctcgggcggtggtgggtcctatgwgctgacwcagccacc

SEQ ID NO: 71 - DNA, 77 bp, Artificial Sequence
  Primer sequence
ggtggttcctctagatcttcctcctctggtggcggtggctcgggcggtggtgggtcttctgagctgactcaggaccc

SEQ ID NO: 72 - DNA, 77 bp, Artificial Sequence
  Primer sequence
ggtggttcctctagatcttcctcctctggtggcggtggctcgggcggtggtgggcacgttatactgactcaaccgcc

SEQ ID NO: 73 - DNA, 77 bp, Artificial Sequence
  Primer sequence
ggtggttcctctagatcttcctcctctggtggcggtggctcgggcggtggtgggcagcytgtgctgactcaatcryc

SEQ ID NO: 74 - DNA, 75 bp, Artificial Sequence
  Primer sequence
ggtggttcctctagatcttcctcctctggtggcggtggctcgggcggtggtgggctgcctgtgctgactcagccc

SEQ ID NO: 75 - DNA, 74 bp, Artificial Sequence
  Primer sequence
ggtggttcctctagatcttcctcctctggtggcggtggctcgggcggtggtgggcagsctgtgctgactcagcc

SEQ ID NO: 76 - DNA, 77 bp, Artificial Sequence
  Primer sequence
ggtggttcctctagatcttcctcctctggtggcggtggctcgggcggtggtgggaattttatgctgactcagcccca

SEQ ID NO: 77 - DNA, 77 bp, Artificial Sequence
  Primer sequence
ggtggttcctctagatcttcctcctctggtggcggtggctcgggcggtggtgggcagrctgtggtgacycaggagcc

SEQ ID NO: 78 - DNA, 77 bp, Artificial Sequence
  Primer sequence
ggtggttcctctagatcttcctcctctggtggcggtggctcgggcggtggtgggcwgccwgkgctgactcagccmcc

SEQ ID NO: 79 - DNA, 48 bp, Artificial Sequence
  Primer sequence
cttgtcgtcatcgtctttgtagtcagaggasggygggaacagagtgac

SEQ ID NO: 80 - DNA, 36 bp, Artificial Sequence
  Primer sequence
atctcagcggccgcactaattaatgtggcctcagga

SEQ ID NO: 81 - DNA, 33 bp, Artificial Sequence
  Primer sequence
cgcagtaagcttgtcgtcatcgtctttgtagtc

SEQ ID NO: 82 - DNA, 51 bp, Artificial Sequence
  Primer sequence
atgtttttggcagtctttacacttctggcactaattaatgtggcctcagga

SEQ ID NO: 83 - DNA, 88 bp, Artificial Sequence
  Primer sequence
gataagatttaaatataaaagatatgcaactagaaaagtcttatcaatctccggatcctcattacttgtcgtcatcgtctttgtagtc

SEQ ID NO: 84 - protein, 308 aa, Saccharomyces cerevisiae
MTMPHRYMFLAVFTLLALINVASGQVQLVQSGAEVKKPGEPLKISCKGSGYSFTSYWIGWVRQMPGKGLEWMGIIYPGDSDTRYSPSFQGQVTISADKSISTAYLQWSSLKASDTAMYYCARLGYYDSSGYYSEDYWGQGTLVTVSSGSASAPTLGGSSRSSSSGGGGTGGGGQPVLTQSSSASASLGASVKLTCTLSSGRSGFAIAWHQQHPEKGPRYLMNVNSDGSHTKGDGIPDRFSGSSSEAERYLTISSLQFDDEVDYYCQTWGTGIWVFGGGTKLTVLRQPKAAPSVTLFPAVLDYKDDDDK

SEQ ID NO: 85 - protein, 24 aa, Saccharomyces cerevisiae
MTMPHRYMFLAVFTLLALINVASG

SEQ ID NO: 86 - protein, 8 aa, Saccharomyces cerevisiae
DYKDDDDK

(19) United States
(12) Patent Application Publication
(10) Pub. No.: US 2021/0214719 A1
(43) Pub. Date: Jul. 15, 2021

(54) A YEAST PHENOTYPIC SCREENING METHOD FOR ISOLATION OF FUNCTIONAL ANTIBODIES AGAINST G-PROTEIN COUPLED RECEPTORS

(71) Applicant: KANGTI PTE. LTD., Singapore (SG)

(72) Inventor: Mingjie CAI, Singapore (SG)

(21) Appl. No.: 17/059,706

(22) PCT Filed: May 31, 2019
(86) PCT No.: PCT/SG2019/050282

§ 371 Date: Nov. 30, 2020

(30) Foreign Application Priority Data
May 31, 2018 (SG) 10201804620V

(51) Int. Cl. C12N 15/10; C07K 16/00; C07K 16/28

(52) U.S. Cl. CPC C12N 15/1037; C07K 2317/14; C07K 16/28; C07K 16/005

(57) ABSTRACT

The present invention provides a method for identifying a functional antibody or antigen-binding protein or a fragment thereof that is capable of binding to, and stimulating the activity of, a target transmembrane protein comprising the steps of providing yeast cells transformed with yeast expression vectors encoding a library of antibodies or antigen-binding proteins or fragments thereof, wherein said yeast cells express said target transmembrane protein, expressing said library of antibodies or antigen-binding proteins or fragments thereof in the yeast cells, wherein said expressed antibodies or antigen-binding proteins or fragments thereof are secreted into the periplasmic space of said yeast cells, incubating the yeast cells in or on a selective medium or under a restrictive temperature, or a combination thereof, and detecting a predetermined phenotype in the yeast cells, wherein detection or manifestation of the predetermined phenotype is indicative of binding of the functional antibody or antigen-binding protein or a fragment thereof to said target transmembrane protein and stimulation of said target transmembrane protein by the functional antibody or antigen-binding protein or a fragment thereof. Antibodies or antibody fragments that are agonists of G-protein coupled receptors identified by the method of the invention are also provided.

### FIELD OF THE INVENTION

The invention is in the field of biotechnology. In particular, the invention is in the field of antibody identification using yeast phenotypic screening.

### BACKGROUND OF THE INVENTION

The G protein-coupled receptors (GPCRs), also known as seven transmembrane proteins, are a unique family of receptors that are as similar structurally as they are diverse functionally. The members of this superfamily are found in all eukaryotic organisms from yeast to human, and all possess a kindred core structure comprising seven membrane-spanning α-helices linked by six protruding interhelical loops with three of them, along with the N-terminus, jutted into the extracellular space, and the other three, along with the C-terminus, exposed to the cytoplasm. The extracellular and intracellular domains of GPCRs contain the ligand interaction site and the association site of the signal transducing machinery, respectively, thus capacitating the receptor to work as a molecular device that detects the environmental signals and passes the information into the cell to commence various corresponding events. Different GPCRs respond to different ligands which are of a wide range of sizes and types including proteins, peptides, lipids, amino acid and nucleic acid derivatives, ions and even photons. Binding of ligands on the extracellular side triggers a profound conformational change in GPCR which in turn prompts the activation of the signaling networks on the cytosolic side of the receptor, leading to such essential physiological processes as the sensing of light, smell, and taste, modulation of the psychological and neurological states, homeostasis of metabolism, regulation of the immune system, and so on. There are at least 800 GPCR genes present in the human genome and many of them have been implicated in diseases including immune and neurological disorders, cancer and inflammation, and metabolic diseases. This receptor family, therefore, has long existed as the most prominent and prolific targets for the drug industry.

In the last few decades, monoclonal antibodies have morphed from being ordinary laboratory reagents and diagnostic tools to constituting a class of novel therapeutics with astonishing performance in the clinics as well as on the pharmaceutical market. The monoclonal antibody technologies, such as that of antibody generation and engineering, abetted by research findings on disease-related biological targets, have essentially changed the landscape of drug discovery. Unlike many synthetic chemical drugs, antibodies are highly specific to their targets, and usually easy to generate. Thus, a potential biological target identified by laboratory studies can be directly and often decisively evaluated for its clinical significance with monoclonal antibodies that activate or inhibit its function in relevant in vitro and in vivo models, provided that the target is accessible to the antibodies in the body. Such target validation studies are customarily more complex, protracted and expensive with small molecule compounds. Monoclonal antibodies also exhibit additional superior attributes over small molecule drugs such as low toxicity and high stability. As a result, after the proof-of-concept is established with antibody interference, the antibody products are able to achieve a higher rate of success in progressing through the clinical trials and approval process, and reach the market in a much shorter time compared to small molecule drugs. Today, antibody drugs, with an annual market value of over $80 billion and projected to grow to $140 billion by 2024, are the preferred form of treatment for varieties of health problems such as cancer and autoimmune diseases, either because they offer better results, or simply because there are no other choices.

It may seem extraordinary that the antibody drugs targeted at GPCRs have been, so far, few and slow in coming, inasmuch as the latter is widely considered to be among the most heavily investigated drug targets in the pharmaceutical industry. This, in fact, reflects the magnitude of the problem faced by researchers attempting to generate clinically effective antibodies against GPCRs. Being seven-transmembrane proteins whose functions are dependent on their elaborate spatial conformations which, in turn, are dependent on their localization in the plasma membrane, GPCRs are inherently intractable to development of functional antibodies. Various strategies have been practiced to battle this challenge. These include stabilizing GPCRs by mutations or modifications, either in vivo or in vitro, to facilitate purification of homogenous and conformationally preserved antigens, immunizing or screening with linear peptides of extracellular loops and terminus, or with the native antigens in the form of whole cells and membrane fractions of transfected cell lines that over-express the target receptor on the cell surface, or of the DNA. Each of these approaches has its own advantages and limitations and none of them is universally applicable. For example, selection of linear antigens, which are commonly a preferred choice, may work well for receptors whose N-terminus participates directly in ligand binding (such as some chemokine receptors), but does not work for the majority of GPCRs. Thermostabilization of receptors by mutations locks a receptor in one conformation that may not be conducive to raising antibodies with receptor modulating activities. The best form of antigen is, of course, the one embedded in the plasma membrane since it is in the native form and is most likely to give rise to functional antibodies. However, since the GPCR proteins expressed in the engineered cell line are still a small minority relative to the endogenous membrane proteins, immunization using whole cells, cell membrane fractions, and DNA, is generally a very inefficient method and requires extensive screening or counter-screening to pinpoint any true GPCR binders. A monoclonal antibody against CCR5, PA14, for example, was originally obtained by whole cell immunization, but not before sieving through some 10,000 hybridomas.

Finding a functional antibody for any target, GPCRs or otherwise, is in most cases a two-step process, as all of the current antibody discovery technologies, including monoclonal or combinatorial library technologies, are based on antigen binding only. Whether the binders also possess a receptor-modulating activity, be it positive or negative, requires further exploration. Again, using the CCR5 example mentioned above, among the six specific CCR5-interacting antibodies identified from 10,000 hybridomas, only one, PA14, was found to be an antagonist, and none was an agonist. Clearly, if sufficient quantities of structurally appropriate antigens were available, finding agonist and antagonist antibodies would certainly have been more probable. Unfortunately, for a large number of GPCRs, this is not the case, and discovery of functional antibodies for many clinically significant GPCRs remains prohibitively difficult and time- and resource-consuming.

Functional antibodies can be directly isolated by a phenotypic screen without the affinity selection step. Although no examples of attaining such antibodies against GPCRs by this approach are known, there are numerous reports of using this approach to find functional antibodies against other receptors and cell surface antigens. For example, a robust phenotypic screen method named “autocrine screen” which employs a combinatorial antibody library carried by a lentiviral vector has successfully yielded agonistic antibodies against a series of growth factor receptors such as erythropoietin (Epo) receptor, thrombopoietin (Tpo) receptor, G-CSF receptor, FGF receptors, etc. However, this mammalian phenotypic screening method is unlikely suitable for use with GPCRs, because, for example, a robust selection scheme like prevention of cell death that applied readily to growth factor receptors may not be possible, or at least not so straightforward, to contrive for the majority of GPCR receptors in a mammalian cell system.

Presently, there is an absence of an efficient and robust method for identifying functional antibodies, particularly the agonist antibodies, to GPCRs. Accordingly, there is a need to develop a fast, reliable, and low cost method that overcomes the disadvantages described above.

### SUMMARY

In one aspect, there is provided a method for identifying a functional antibody or antigen-binding protein or a fragment thereof that is capable of binding to, and stimulating the activity of, a target transmembrane protein comprising:
a) providing yeast cells transformed with yeast expression vectors encoding a library of antibodies or antigen-binding proteins or fragments thereof, wherein said yeast cells express said target transmembrane protein;
b) expressing said library of antibodies or antigen-binding proteins or fragments thereof in the yeast cells, wherein said expressed antibodies or antigen-binding proteins or fragments thereof are secreted into the periplasmic space of said yeast cells;
c) incubating the yeast cells in or on a selective medium or under a restrictive temperature, or a combination thereof; and
d) detecting a predetermined phenotype in the yeast cells, wherein manifestation of the predetermined phenotype is indicative of binding of the functional antibody or antigen-binding protein or a fragment thereof to said target transmembrane protein and stimulation of the activity of said target transmembrane protein.

In one aspect, there is provided an antibody or antigen-binding protein or a fragment thereof that is the agonist of a target transmembrane protein identified according to the method as disclosed herein, wherein the target transmembrane protein is the human G-protein coupled receptor (GPCR) GLP-1R, or a yeast GPCR, the α-factor receptor Ste2p.

In one aspect, there is provided an antibody or antigen-binding protein or a fragment thereof identified by the method as disclosed herein comprising (i) a heavy chain variable domain comprising a VHCDR1 having the amino acid sequence GYSFTSYW (SEQ ID NO: 1); a VHCDR2 having the amino acid sequence IYPGDSDT (SEQ ID NO: 2); and a VHCDR3 having the amino acid sequence CARLGYYDSSGYYSEDY (SEQ ID NO: 3); and (ii) a light chain variable domain comprising a VLCDR1 having the amino acid sequence SGRSGFA (SEQ ID NO: 4), a VLCDR2 having the amino acid sequence VNSDGSH (SEQ ID NO: 5), and a VLCDR3 having the amino acid sequence QTWGTGIWV (SEQ ID NO: 6).

### Definitions

The following are some definitions that may be helpful in understanding the description of the present invention. These are intended as general definitions and should in no way limit the scope of the present invention to those terms alone, but are put forth for a better understanding of the following description.

The phrase “antibodies or antigen-binding proteins or fragments thereof” as used herein refers to protein molecules encompassing natural and engineered antibodies, antibody fragments and proteins not related to antibodies and their fragments, such as protein domains and oligo-peptides, that are capable of binding to an epitope of a target antigen in vivo and in vitro in a selective manner.

The term “antigen” as used herein refers to proteins of interest that are capable of being recognized and reacted to by antibodies in vivo and in vitro in a selective manner. Preferred antigens encompass proteins that reside in the plasma membrane of a cell and act as sensors, receptors, or transporters for specific substances in the extracellular space. Further preferred antigens are the family of transmembrane proteins known as G-protein coupled receptors.

The term “antibody” is used herein in the broadest sense to refer to various forms of immunoglobulin molecules and their immunologically active fragments, natural or engineered, including monoclonal antibodies, recombinant antibodies, polyclonal antibodies, chimeric antibodies, humanized antibodies, bispecific antibodies, heteroconjugate antibodies, chimeric antigen receptors (CAR), a single variable domain, a domain antibody, antigen-binding fragments, immunologically effective fragments, diabodies, multispecific antibodies, human antibodies, camelid antibodies, single-chain variable fragments (scFv), disulfide-linked variable fragments (sdFv), Fab fragments, F (ab′) fragments, and anti-idiotypic (anti-Id) antibodies (including, e.g., anti-Id antibodies to antibodies of the invention) and epitope-binding fragments of any of the above. The immunologically active antibody fragments may also be fused to any polypeptide fusion partner including the Fc region or a fragment thereof. Examples of Fc variant-fusions include scFv-Fc fusions, variable region (e.g., VL and VH)-Fc fusions, or scFv-scFv-Fc fusions. Antibodies can be of any type (e.g., IgG, IgE, IgM, IgD, IgA and IgY), class (e.g., IgG₁, IgG2, IgG3, IgG4, IgA1 and IgA2) or subclass.

The phrase “single variable domain” refers to a variable domain of an antibody (for example, V_{H}, V_{HH}, V_{L}) that binds to an antigen or epitope independently of a different variable region or domain. A single variable domain may include a complete antibody variable domain or a modified variable domain. Examples of modified variable domains include ones in which one or more residues in the antigen-interacting motifs are changed to other sequences, and ones which are truncated or comprise N- or C-terminal extensions.

As used herein the term “domain” refers to a folded protein structure which has distinctive structural characteristics of its own. Generally, domains are responsible for discrete functional properties of proteins, and in many cases may be added, removed or transferred to other proteins without loss of function of the remainder of the protein and/or of the domain.

An antigen-binding fragment or an antigen-binding domain may be transferred to other appropriate proteins or domains by means of arrangement of one or more CDRs on non-antibody protein scaffolds to generate protein molecules that acquire the binding activity to a same target antigen. Examples of scaffold proteins include CTLA-4, lipocalin, SpA, an Affibody, an avimer, GroE1, transferrin, GroES and fibronectin/adnectin.

An antigen-binding fragment or an immunologically effective fragment may comprise partial heavy and/or light chain variable sequences. Fragments are at least 5, 6, 8 or 10 amino acids in length. Alternatively the fragments are at least 15, at least 20, at least 50, at least 75, or at least 100 amino acids in length.

“CDRs” refer to the complementarity-determining region of an antibody or the fragment thereof. These are the hypervariable regions of immunoglobulin heavy and light chains. There are three heavy chain and three light chain CDRs (or CDR regions) in the variable portion of an immunoglobulin. Thus, “CDRs” as used herein refers to any or all of the three heavy chain CDRs and the three light chain CDRs.

An antigen-binding protein may also be a polypeptide or oligo-peptide. An oligo-peptide may comprise between 2 to 50 amino acids, and preferably more than 10 amino acids. For example, an oligo-peptide may be a dipeptide, a tripeptide, a tetrapeptide or a pentapeptide. A polypeptide may comprise more than 50 amino acids.

The term “functional” when used in the context of antibodies or antigen-binding proteins or fragments thereof refers to the ability of such molecules to bind to their target antigen and elicit a biological response mediated by said target antigen. “Functional” is also used to describe antibodies or antigen-binding proteins or fragments thereof that possess either native biological activity or any specific activity unidentified before. Examples of functional antibodies or antigen-binding proteins or fragments thereof include such antibodies or antigen-binding proteins or fragments thereof that have the ability to bind to membrane-localized receptors and stimulate the receptor-mediated signal transduction pathway. A functional antibody or antigen-binding protein or a fragment thereof may be an agonist or an antagonist.

The term “library” refers to combinatorial DNA libraries consisting of a mixture of heterogeneous polynucleotides that encode a heterogeneous population of polypeptides or oligo-peptides (e.g., a heterogeneous population of antibody polypeptides or the polypeptides or oligo-peptides not derived from antibodies) from which the antibodies or antibody fragments or polypeptides or oligo-peptides possessing desired structural or functional properties are selected. Sequence differences between library members are responsible for the diversity of the library. The library can take the form of a simple mixture of polynucleotides, or can be in the form of a population of organisms or cells, for example yeast cells and the like, with each cell containing one or more copies of an expression vector comprising divergent polynucleotide sequences encoding antibodies or antibody fragments or polypeptides or oligo-peptides not derived from antibodies that can be expressed in the yeast cells. Thus, the population of host cells represents a heterogeneous population of antibodies or polypeptides encoded in the said library. Examples of combinatorial libraries include combinatorial antibody libraries encompassing single-chain and single domain libraries (e.g., scFv and VHH), Fab libraries, and full-length antibody libraries, and combinatorial peptide libraries. Combinatorial antibody libraries may further include immune libraries, naïve libraries, semi-synthetic libraries, synthetic libraries and combinations thereof. Combinatorial peptide libraries may further include random or partially random polypeptide or oligo-peptide libraries.

The term “library” may be used interchangeably with the term “repertoire”.

As used herein, the term “polynucleotide encoding an antibody or a fragment thereof” encompasses a composition of polynucleotides comprising one or more polynucleotide chains encoding the individual polypeptide chains of said antibody or a fragment thereof.

As used herein, “vector” refers to any element capable of serving as a vehicle of genetic transfer, gene expression, or replication or integration of a foreign polynucleotide in a host cell. A vector can be an artificial chromosome or plasmid, and can be integrated into the host cell genome or exist as an independent genetic element (e.g., episome, plasmid). A vector is capable of directing expression of a library of polypeptides in the host cell. Vectors of the present invention include yeast expression vectors, 2μ vectors and centromere-containing vectors.

A “shuttle vector” (or bi-functional vector) is known in the art as any vector that can replicate in more than one species of organism. For example, a shuttle vector that can replicate in both Escherichia coli (E. coli) and Saccharomyces cerevisiae (S. cerevisiae) can be constructed by linking sequences from an E. coli plasmid with sequences from the yeast 2μ plasmid.

Polynucleotides may be incorporated into expression vectors, in order to allow expression of the polypeptides encoded by the polynucleotides.

As used herein, the term “transmembrane domain” refers to the domain of a peptide, polypeptide or protein that is capable of spanning the plasma membrane of a cell.

As used herein, the term “promoter” is intended to refer to a region of DNA that initiates transcription of a particular gene.

As used herein, the term “detectable label” or “reporter” refers to a detectable marker such as a gene or phenotype that is expressed only when a signal transduction pathway is activated.

As used herein, the term “secretion signal” or “secretory signal peptide” refers to a short polypeptide sequence (usually 16-30 amino acids) located at the N-terminus of a protein that directs the translocation of the said protein through the secretory pathway to cellular destinations such as endoplasmic reticulum, Golgi, endosomes, plasma membrane, or the extracellular space.

As used herein, the terms “periplasmic space” or “periplasm” refer to the space between the plasma membrane and the cell wall of a yeast cell.

As used herein, the term “about”, in the context of concentrations of components of the formulations, typically means+/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

### BRIEF DESCRIPTION OF THE DRAWINGS

The invention will be better understood with reference to the detailed description when considered in conjunction with the non-limiting examples and the accompanying drawings, in which:

FIG. 1 is a diagram of the yeast phenotypic screening system for isolation of agonistic antibodies against GPCRs. The screen illustrated here uses an scFv library but it is also suitable for other forms of libraries including other antibody libraries such as an Fab library, as well as general peptide libraries. The antibody library is introduced into yeast cells by either gap-repair recombination between a gapped vector and a PCR-generated antibody repertoire, or in the form of pre-made library as single-molecule plasmids. Once inside the cell, the antibody is expressed and secreted into the periplasmic space between the plasma membrane and the cell wall where it is mostly retained due to its size. The screening will identify the antibody that can recognize the membrane-located GPCR and trigger the activation of the pheromone signaling pathway which leads to the expression of CDC26, neoR, or zeoR genes, enabling cell growth at 37° C., or on the antibiotic-containing media, or both.

FIG. 2 shows plasmid maps. The scFv library is inserted at the NotI-SacII site of pKT047 or pKT103, or at the NotI-AscI site of pKT131, replacing the CH1 sequence and the TEMP sequence, respectively. The scFv inserted in pKT047 is bestowed with a GPI anchor and a V5 epitope tag C-terminal to the scFv sequence, and those inserted in pKT103 has no membrane anchor but contain a FLAG tag at the C-terminus provided by the PCR primer. Similarly, the scFv inserted in pKT131 is anchor-less and contain a 6×HIS tag at the C-terminus. The maps were not drawn to scale.

FIG. 3 shows the GPCR-dependent high temperature survival of YKT099 cells. Late log phase cultures of YKT099 (pFUS2::CDC26 pFIG1::neoR) and YKT112 (pFUS2::CDC26 pFIG1::neoR ste2Δ) growing in YPD at 28° C. were diluted with medium and 250 μl of approximately 10³ fold diluted cultures were spread on YPD plates supplemented with or without G418 or α-factor. The concentrations of G418 and α-factor were 20 μg/ml (G20) and 80 ng/ml, respectively. The plates were then placed at 37° C. A control plate for each strain was incubated at 28° C. for the measurement of total cells plated on each plate. The plates were scanned after 3 days of incubation.

FIG. 4 shows the GPCR-dependent G418 resistance of YKT099 cells. Late log phase cultures of different strains of cells growing in YPD at 28° C. were streaked onto YPD plates that contained G418 at the indicated concentrations (G50=50 μg/ml, etc.). In parallel, they were also streaked onto a set of G418-containing plates that were supplemented with α-factor at 80 ng/ml (bottom row). All plates were then placed at 30° C., except the drug-free control plate which was incubated at 28° C. The plates were scanned after 3 days of incubation.

FIG. 5 shows the GPCR-dependent Zeocin resistance of YKT113 cells.

Late log phase cultures of different strains of cells growing in YPD at 28° C. were streaked onto YPD plates that contained Zeocin at the indicated concentrations (Z10=10 μg/ml, etc.). In parallel, they were also streaked onto a set of Zeocin-containing plates that were supplemented with α-factor at 80 ng/ml (bottom row). All plates were then placed at 28° C. The plates were scanned after 3 days of incubation.

FIG. 6 shows the isolation of human antibody fragments that could stimulate the yeast GPCR signaling pathway. YKT099 cells transformed with the control vector pKT103 and five scFv-containing plasmids (Q5, W4, W5, W8 and W19) that were isolated from the screen as positives were cultured at 28° C. in SC-Leu medium to late log phase. The undiluted cultures were streaked onto SC-Leu plates containing G418 at the indicated concentrations (G20=20 μg/ml, etc.) and the plates were incubated at the indicated temperatures. The plates were scanned after 3 days of incubation.

FIG. 7 shows the amino acid sequence of Q5. The amino acid sequence of Q5 is shown in the single letter code. FLO1SP: signal peptide of Flo1p; HC: heavy chain; LC: light chain; CDR: complementariy-determining region; FLAG: the FLAG epitope tag. The CDR regions are shown in red. The germline alleles were identified by IMGT, the international ImMunoGene Tics information system (www.imgt.org).

FIG. 8 shows that Q5 supports YKT099's growth at high temperature and in the presence of G418 by stimulating the yeast GPCR signaling pathway. YKT099 cells containing the Q5 expression plasmid pKT112 and the control vector pKT103 were cultured to late log phase in the SC-Leu medium at 28° C. The undiluted cultures were streaked onto SC-Leu plates containing G418 at the indicated concentrations (G100=100 μg/ml, etc.) and allowed to grow at 30° C. A control plate containing no drug was incubated at 28° C. (A). In parallel, the cultures were diluted approximately 10³ fold and 250 μl of the each diluted culture were spread on SC-Leu plates with or without G418 and the plates were incubated at the indicated temperatures (B). YKT112 containing pKT112 was similarly cultured and plated for comparison (B). All plates were scanned after 3 days of incubation.

FIG. 9 shows the stimulation of pFIG1::GFP expression in YKT099 cells by Q5. YKT099 cells containing the Q5 expression plasmid (pKT112) and the control vector (pKT103) were cultured in SC-Leu medium at 28° C. for 48 h. The cultures were then diluted 10 fold with the medium and allowed to grow at 28° C. for another 4 h before collected for microscopy examination.

FIG. 10 shows the stimulation of pFIG1::LacZ expression in YKT109 cells by Q5. YKT109 cells, which has the FIG1 promoter-driven β-galactosidase gene (pFIG1::lacZ) integrated into its genome, were transformed with the control vector, pKT103, the Q5 expression plasmid, pKT112, and a plasmid containing an scFv sequence randomly picked up from the library transformants, G3. The transformed cells were patched onto a SC-Leu plate and allowed to grow for 2 days. The patches grown on the plate were then lifted onto a filter paper which was used to perform the β-galactosidase lift assay. The color development was allowed for 2 hrs.

FIG. 11 shows the stimulation of pheromone-inducible gene expression in YKT099 cells by Q5. RT-qPCR was employed to measure the expression of the native FUS1 and the engineered pFIG1::neoR genes, both of which are pheromone inducible, in YKT099 cells that contain the Q5 expression plasmid pKT112 or the control vector pKT103. The primers used to detect the transcripts of these genes, as well as that of the ACT1 gene which served as the internal control, were given in materials and methods.

FIG. 12 shows the cell surface localization of Q5-GFP. BJ5465 cells containing pKT112 (Q5) or pKT119 (Q5-GFP) were cultured in SC-Leu medium at 28° C. to the late log phase. The cultures were diluted 5 fold and allowed to grow for additional 5-6 hr at 28° C. before collected for microscopic examination.

FIG. 13 compares the ability of Q5 with or without a GPI anchor element to stimulate the signaling pathway. YKT099 cells containing Q5 expressed either as a free form (pKT112) or with a GPI anchor element attached to the C-terminus (pKT113) were cultured to late log phase in SC-Leu medium at 28° C. The cultures were diluted with medium and 250 μl of each diluted cultures were spread on SC-Leu plates containing G418 at the indicated concentrations (G300=300 μg/ml, etc.) and the plates were incubated at various temperatures as labeled in the figure. The plates were scanned after 3 days of incubation.

### DETAILED DESCRIPTION OF THE PRESENT INVENTION

In a first aspect the present invention refers to a method for identifying a functional antibody or antigen-binding protein or a fragment thereof that is capable of binding to, and stimulating the activity of, a target transmembrane protein comprising:
a) providing yeast cells transformed with yeast expression vectors encoding a library of antibodies or antigen-binding proteins or fragments thereof, wherein said yeast cells express said target transmembrane protein;
b) expressing said library of antibodies or antigen-binding proteins or fragments thereof in the yeast cells, wherein said expressed antibodies or antigen-binding proteins or fragments thereof are secreted into the periplasmic space of said yeast cells;
c) incubating the yeast cells in or on a selective medium or under a restrictive temperature, or a combination thereof; and
d) detecting a predetermined phenotype in the yeast cells, wherein manifestation or detection of the predetermined phenotype is indicative of binding of the functional antibody or antigen-binding protein or a fragment thereof to said target transmembrane protein and stimulation of the activity of said target transmembrane protein by the functional antibody or antigen-binding protein or a fragment thereof.

In one embodiment, the target transmembrane protein is a G-protein coupled receptor, or GPCR.

In another embodiment, the functional antibody or antigen-binding protein or a fragment thereof is an agonist of GPCR.

In one embodiment, the yeast cells in step a) may comprise one or more reporter genes, wherein expression of the one or more reporter genes is inducible by activation of a GPCR-mediated signal transduction pathway. In a preferred embodiment, the GPCR-mediated signal transduction pathway is based on the yeast mating pheromone signaling pathway. It will generally be understood that the one or more reporter genes may be induced by the yeast mating pheromone signaling pathway. In some embodiments, the reporter gene may be an antibiotic resistance gene, a temperature-sensitivity-complementing gene, a fluorescent protein gene, or combinations thereof. Suitable antibiotic resistance genes, temperature-sensitivity-complementing genes and fluorescent genes that may be employed in the present invention would be known to a person skilled in the art. In a preferred embodiment, the antibiotic resistance gene is the G418-resistance gene (neoR) or the zeocin resistance gene (zeoR). In another preferred embodiment, the temperature-sensitivity-complementing gene is CDC26. In yet another preferred embodiment, the fluorescent protein genes are GFP, RFP, YFP, BFP, or CFP. In yet another preferred embodiment, the reporter gene is LacZ.

In one embodiment, the predetermined phenotype is cell growth in the presence of one or more antibiotics due to expression of the reporter antibiotic resistance genes, cell growth at a restrictive temperature due to expression of the reporter temperature-sensitivity-complementing genes, generation of fluorescence due to expression of the reporter fluorescent protein genes, or combinations thereof. In a preferred embodiment, the reporter antibiotic resistance genes are the G418 resistance gene neoR and the Zeocin resistance gene zeoR. In another preferred embodiment, the reporter temperature-sensitivity-complementing gene is CDC26 and the restrictive temperature may be between about 30° C. and about 40° C. In yet another preferred embodiment, the fluorescent protein genes are GFP, RFP, YFP, BFP, or CFP.

In some embodiments, the target transmembrane protein in step a) is a G-protein coupled receptor, or GPCR. In one embodiment, the target transmembrane protein is the Ste2 protein of the yeast Saccharomyces cerevisiae. In another embodiment, the target transmembrane protein is the human GPCR receptor GLP-1R.

In one embodiment, the antigen-binding protein is an antibody-related molecule capable of interacting with target antigens and possesses one or more complementarity-determining regions (CDRs). In another embodiment, the antigen-binding protein is a polypeptide or oligo-peptide capable of interacting with target antigens and comprises of an amino acid sequence not derived from an antibody. In a preferred embodiment, the antigen-binding protein is a single-chain variable fragment (scFv) or an antigen-binding fragment (Fab).

In one embodiment, the functional antibody or antigen-binding protein or a fragment thereof is a G-protein coupled receptor agonist.

In one embodiment, the target transmembrane protein (e.g. a GPCR) may be expressed on the plasma membrane of the yeast cells. It will generally be understood that binding of an agonist to the target transmembrane protein stimulates the cellular activity of said target transmembrane protein and elicits downstream cellular responses such as a signal transduction pathway in the case of GPCR which leads to the expression of the one or more reporter genes. Accordingly, in one embodiment, the expression of the one or more reporter genes is induced by binding of an agonist to the target transmembrane protein.

The method of the invention also involves expressing a library of antibodies or antigen-binding proteins or fragments thereof in yeast cells, particularly in the periplasmic space of the yeast cells. The library of antibodies or antigen-binding proteins or fragments thereof may comprise single-chain variable fragment (scFv), disulfide-linked variable fragment (sdFv), Fab fragments and F (ab′) fragments, V_{H}dAb or cFvs. In one embodiment, the library of antibodies or antigen-binding proteins or fragments thereof may comprise single-chain variable fragment (scFv), single domain (VHH), Fab fragments, polypeptide or oligo-peptides. In another embodiment, the peptide or oligo-peptide library is a random or partially random peptide library that is not derived from antibody sequences.

In another embodiment, the nucleic acid sequence encoding the antibodies or antigen-binding proteins or fragments thereof further comprises a secretion signal sequence, which facilitates the localization of the said antibodies or antigen-binding proteins or fragments thereof to the periplasmic space of the yeast cells.

The secretion signal sequence may be that of the yeast mating pheromone alpha factor (α factor) or that of the Flo1p, Suc2p, or Aga2p. In a preferred embodiment, the secretion signal sequence may be encoded by the yeast mating pheromone alpha factor gene MFα1 or the FLO1 gene.

In some embodiments, the antibodies or antigen-binding proteins or fragments thereof may be coupled to an anchor element to be retained in the periplasmic space of the yeast cells. In another embodiment, the antibodies or antigen-binding proteins or fragments thereof may be fused with a transmembrane domain to be retained in the periplasmic space of the yeast cells. In a preferred embodiment, the anchor protein may be a glycosylphosphatidylinositol (GPI)-attachment protein or a transmembrane domain. In one embodiment, the antibodies or antigen-binding proteins or fragments thereof may be further linked to a tag. Examples of such tags include but are not limited to polyhistidine, gluthathione S-transferase, calmodulin binding peptide, intein-chitin binding domain, streptabidin/biotin based tags, tandem affinity purification tags. In a preferred embodiment, the tag is an epitope tag. In a further preferred embodiment, the epitope tag is a FLAG tag or a 6×HIS tag.

Assembly of the library may be performed using methods known in the art. The library of antibodies or antigen-binding proteins or fragments thereof may be inserted into a plasmid vector such as pKT047, pKT103, or pKT131. In one embodiment, the nucleic acid sequence encoding the antibodies or antigen-binding proteins or fragments thereof is operably linked to a yeast promoter. In a preferred embodiment, the promoter may be a constitutive promoter such as the TEF1 promoter, or an inducible promoter such as the GAL1 promoter.

It will generally be understood that the library may be constructed from a variety of cell sources. For example, the library may be constructed from animal bone marrow cells, spleen and lymph node cells, or circulating white blood cells. In a preferred embodiment, the library may be constructed from human peripheral blood mononuclear cells.

The library may have a diversity or complexity in the range of 10⁵ to 10¹², for example, 10⁵, 10⁶, 10⁷, 10⁸, 10⁹, 10¹⁰, 10¹¹, or 10¹². In a preferred embodiment, the diversity of the library of single-chain variable fragment (scFv), single domain (VHH) or Fab fragments is greater than 10⁷. In another preferred embodiment, the diversity of the polypeptides or oligo-peptides is greater than 10⁵. These numbers refer to the diversity of the original library that has not gone through any forms of target-directed enrichment.

In some embodiments, the oligo peptide library comprises peptides longer than 5 amino acids.

The yeast cells of the present invention may be transformed by methods generally known in the art. In one embodiment, the yeast cells may be transformed by gap-repair transformation. In other embodiments, transformation of yeast is done with a pre-made library in a plasmid form by electroporation or the heat-shock method.

The yeast cells of the present invention may be budding or filamentous yeast cells. Examples of yeast cells include but are not limited to Saccharomyces cerevisiae, Schizosaccharomyces pombe, Candida albicans, Pichia pastoris and Aspergillus species. In one embodiment, the yeast may be Saccharomyces cerevisiae, Schizosaccharomyces pombe or Pichia pastoris. In a preferred embodiment, the yeast is Saccharomyces cerevisiae or Schizosaccharomyces pombe.

Yeast cells of the present invention may be cultured using media and conditions generally known in the art. Culture media and culture conditions may be modified to select for one or more predetermined phenotypes. Selective media may comprise one or more antibiotics. In one embodiment, the selective media may comprise zeocin and/or G418 antibiotics. Selective media may also be at a temperature range of about 25-40° C., for example, about at least about 25.5° C., about 26° C., about 26.5° C., about 27° C., about 27.5° C., about 28° C., about 28.5° C., about 29° C., about 29.5° C., about 30° C., about 30.5° C., about 31° C., about 31.5° C., about 32° C., about 32.5° C., about 33° C., about 33.5° C., about 34° C., about 34.5° C., about 35° C., about 35.5° C., about 36° C., about 36.5° C., about 37° C., about 37.5° C., about 38° C., about 38.5° C., about 39° C., about 39.5° C. and about 40° C. In a preferred embodiment, the selective media is at a temperature of between about 30° C. to about 40° C.

In one embodiment, the functional antibody or antigen-binding protein or a fragment thereof is a monoclonal, recombinant, polyclonal, chimeric, humanised, bispecific and heteroconjugate antibodies, a single variable domain, a domain antibody, antigen-binding fragments, immunologically effective fragments, single-chain variable fragments, a single chain antibody, a univalent antibody lacking a hinge region, a minibody, a diabody, a polypeptide or oligo-peptide comprising an amino acid sequence not derived from an antibody.

The present invention also provides an antibody or antigen-binding protein or a fragment thereof that is an agonist of a GPCR identified according to the method of the present invention, wherein the GPCR is human GLP-1R, or the yeast α-factor receptor Ste2p.

The present invention also provides an antibody or antigen-binding protein identified by the method as disclosed herein comprising (i) a heavy chain variable domain comprising a VHCDR1 having the amino acid sequence GYSFTSYW (SEQ ID NO: 1); a VHCDR2 having the amino acid sequence IYPGDSDT (SEQ ID NO: 2); and a VHCDR3 having the amino acid sequence CARLGYYDSSGYYSEDY (SEQ ID NO: 3); and (ii) a light chain variable domain comprising a VLCDR1 having the amino acid sequence SGRSGFA (SEQ ID NO: 4), a VLCDR2 having the amino acid sequence VNSDGSH (SEQ ID NO: 5), and a VLCDR3 having the amino acid sequence QTWGTGIWV (SEQ ID NO: 6).

In one embodiment, the antibody or antigen-binding protein comprises a heavy chain variable region which comprises the amino acid sequence

(SEQ ID NO: 7)
QVQLVQSGAEVKKPGEPLKISCKGSGYSFTSYWIGWVRQMPGKGLEWMGI
IYPGDSDTRYSPSFQGQVTISADKSISTAYLQWSSLKASDTAMYYCARLG
YYDSSGYYSEDYWGQGTLVTVSSGSASAPTL,

and a light chain variable region which comprises the amino acid sequence

(SEQ ID NO: 8)
QPVLTQSSSASASLGASVKLTCTLSSGRSGFAIAWHQQHPEKGPRYLMNV
NSDGSHTKGDGIPDRFSGSSSEAERYLTISSLQFDDEVDYYCQTWGTGIW
VFGGGTKLTVLRQPKAAPSVTLFPAVL.

It will be generally understood that variation in the CDR, variable heavy chain and variable light chain sequences can exist. In some embodiments, the antibody or antigen-binding protein or a fragment thereof comprises heavy and light chain CDR regions that are about 80%, about 85%, about 90%, about 95%, about 96%, about 97%, about 98% or about 99% identical to the heavy and light chain CDR regions of (i) and (ii).

In other embodiments, the antibody or antigen-binding protein, or a fragment thereof comprises a heavy chain variable region and a light chain variable region that are about 80%, about 85%, about 90%, about 95%, about 96%, about 97%, about 98% or about 99% identity to the amino acid sequence set forth in SEQ ID NO: 7 and 8 respectively.

As used herein, the percent homology between two amino acid sequences is equivalent to the percent identity between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### EXPERIMENTAL SECTION

Non-limiting examples of the invention and comparative examples will be further described in greater detail by reference to specific Examples, which should not be construed as in any way limiting the scope of the invention.

Materials and Methods

Yeast Strains, Vectors, and Growth Conditions

All yeast strains used in this study were derived from BJ5465, and their genotypes were listed in Table 1. Gene deletions and integrations were done using a recyclable URA3 cassette. For construction of YKT068, the SST2, FAR1, and CDC26 genes in BJ5465 were deleted sequentially, and a 650 bp fragment of the CDC26 gene from the Start codon was amplified from wildtype genomic DNA by PCR and cloned into an integration vector and inserted immediately downstream of the FUS2 promoter in the genome. This was followed by inserting a GFP coding sequence preceded by the FIG1 promoter region (−600 to −1) into the HIS3 sequence which was at the pep4 locus in BJ5465. The coding sequence of the STE2 gene in YKT068 was additionally deleted to make YKT069. The promoter region of STE2, however, was not affected by the deletion. The genomic coding sequence of FIG1 in YKT068 was replaced by the LacZ coding sequence to make YKT109. YKT099 and YKT112 were made from YKT068 and YKT069, respectively, by inserting a FIG1 promoter driven-neomycin resistance gene (neoR) at the ADE2 locus. Similarly, YKT113 and YKT114 were made from YKT068 and YKT069, respectively, by inserting a FIG1 promoter driven-Zeocin resistance gene (Sh ble, or zeoR) at the ADE2 locus.

TABLE 1
summarizes the yeast strains generated in this study.

| Strain | Genotype |
|---|---|
| (BJ5465) | MATa ura3-52 trp1 leu2-Δ1 his3-Δ200 prb1-Δ1.6R can1 |
| | pep4::HIS3 |
| YKT068 | MATa ura3-52 trp1 leu2-Δ1 his3-Δ200 prb1-Δ1.6R can1 |
| | pep4::his3::pFIG1::GFP sst2Δfar1Δ cdc26Δ pFUS2::CDC26 |
| YKT069 | MATa ura3-52 trp1 leu2-Δ1 his3-Δ200 prb1-Δ1.6R can1 |
| | pep4::his3::pFIG1::GFP sst2Δfar1Δ cdc26Δ ste2Δ |
| | pFUS2::CDC26 |
| YKT099 | MATa ura3-52 trp1 leu2-Δ1 his3-Δ200 prb1-Δ1.6R can1 |
| | pep4::his3::pFIG1::GFP sst2Δfar1Δ cdc26Δ ade2:: |
| | pFIG1::neoR pFUS2::CDC26 |
| YKT106 | MATa ura3-52 trp1 leu2-Δ1 his3-Δ200 prb1-Δ1.6R can1 |
| | pep4::his3::pFIG1::GFP sst2Δfar1Δ cdc26Δ pFUS2::CDC26 |
| | ade2::pFIG1::neoR ste2Δ pSTE2::GLP-1R |
| YKT109 | MATa ura3-52 trp1 leu2-Δ1 his3-Δ200 prb1-Δ1.6R can1 |
| | pep4::his3::pFIG1::GFP sst2Δfar1Δ cdc26Δ pFUS2::CDC26 |
| | pFIG1::LacZ |
| YKT112 | MATa ura3-52 trp1 leu2-Δ1 his3-Δ200 prb1-Δ1.6R can1 |
| | pep4::his3::pFIG1::GFP sst2Δfar1Δ cdc26Δ ste2Δ |
| | ade2::pFIG1::neoR pFUS2::CDC26 |
| YKT113 | MATa ura3-52 trp1 leu2-Δ1 his3-Δ200 prb1-Δ1.6R can1 |
| | pep4::HIS3 sst2Δ far1Δ cdc26Δ ade2::pFIG1::zeoR |
| | pFUS2::CDC26 |
| YKT114 | MATa ura3-52 trp1 leu2-Δ1 his3-Δ200 prb1-Δ1.6R can1 |
| | pep4::HIS3 sst2Δ far1Δ cdc26Δ ste2Δ ade2::pFIG1::zeoR |
| | pFUS2::CDC26 |

To create the GLP-1R expression strains, the last 15 nucleotides of the coding sequence before the stop codon of the GPA1 gene in the chromosomes of YKT069 was changed from AAAATTGGTATTATA (SEQ ID NO: 9) to CAATACGAACTCTTA (SEQ ID NO: 10) by direct replacement using a PCR product. The resulting strain was used for integration of a cloned DNA construct containing the pre-pro alpha factor signal peptide (PPASP) joined to the N-terminus of human GLP-1R without its native signal peptide sequence, and followed by the TEF1 terminator sequence. The integration was directed to occur at the ste2 locus in such a way that the start codon of PPASP-GLP-1R sequence was placed immediately downstream of the STE2 promoter. The resulting strain was subsequently integrated with the FIG1 promoter driven-neoR at the ADE2 locus to generate YKT106.

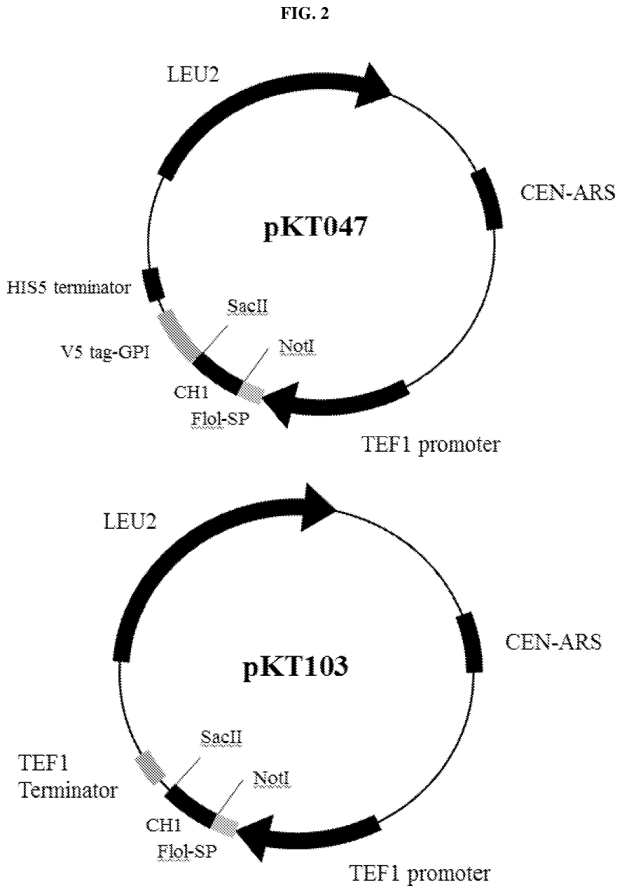

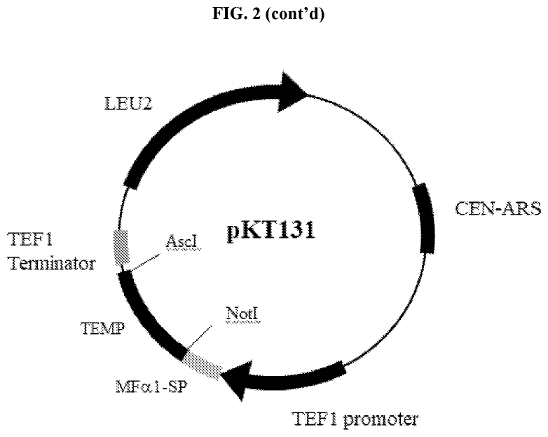

All vectors used in this study were derived from the common shuttle vector pRS315, and those with the prefix of pKT were listed in Table 2. To make pKT047, the oligonucleotide sequence encoding the Flo1p signal peptide (Flo1SP) with a PmeI site added immediately upstream of the ATG codon was cloned into the Spe-NotI site of pRS315 with the NotI site downstream of the SP sequence to generate pRS315-Flo1SP. A 490 bp DNA fragment of the TEF1 promoter (pTEF1) was obtained by PCR and cloned into SmaI-Spe site of pRS315-Flo1SP, placing pTEF1 immediately upstream of Flo1SP to generate pTEF1-Flo1SP. pKT047 was completed by cloning a chemically synthesized DNA fragment comprising a 342 bp CH1 domain, followed by a SacII restriction site, a V5 epitope tag, a 171 bp GPI anchor from Flo1p with a flexible linker, and a 146 bp HISS terminator sequence, into the NotI-SacI site of pTEF1-Flo1SP (FIG. 2). To generate pKT103, the SacII-SacI fragment of pKT047, containing V5 tag-GPI anchor-HISS terminator, was replaced by a 310 bp TEF1 terminator (FIG. 2). pKT131 was made by replacing the Flo1 signal peptide coding sequence in pKT103 with a 267 bp fragment containing the signal peptide of pre-pro-α-factor.

TABLE 2
summarizes the plasmids generated in this study.

| Name | Originated from | Description |
|---|---|---|
| pKT047 | pRS315 | scFv library expression vector |
| | | with a GPI anchor. |
| pKT103 | pRS315 | scFv library expression vector |
| | | without a GPI anchor. |
| pKT112 | pKT103 | Q5 in pKT103. |
| pKT113 | pKT047 | Q5 in pKT047. |
| pKT119 | pKT112 | Q5 in pKT112 fused with GFP |
| | | at the C-terminus. |
| pKT131 | pKT103 | scFv library expression vector |
| | | with MFα1 signal peptide. |

Rich (YPD), synthetic complete (SC), and dropout media were prepared according to the standard methods. Antibiotics were added to the medium shortly after autoclaving. The α-factor containing plates were made by spreading aqueous α-factor solution on the YPD plates which were estimated to contain 25 ml of medium per plate. The temperature-sensitive mutant was propagated at the permissive temperature of 28° C. and analysed at 37° C. or as indicated in the text.

RT-qPCR

The total RNA was isolated from yeast cells using QIAGEN RNeasy minikit following the instruction of the manufacturer. cDNA was synthesized using the iScript reverse transcription supermix for RT-qPCR (Bio-Rad). RT-PCR reactions were performed using SsoAdvanced™ Universal SYBR® Green Supermix and the CFX Connect™ Real-Time PCR Detection System from Bio-Rad. The amplification conditions was 30 sec at 95° C., followed by 40 cycles of 5 sec at 95° C. and 30 sec at 60° C. The primers used to detect the expression of ACT1 (as the internal control), FUS1 and neoR were: ACT1-RT-5′: 5′-ATTCTGAGGTTGCTGCTTTGG-3′ (SEQ ID NO: 11), ACT1-RT-3′: 5′-TGTCTTGGTCTACCGACGATAG-3′ (SEQ ID NO: 12); FUS1-RT-5′: 5′-TCGCCGCAATGTCTACTACC-3′ (SEQ ID NO: 13), FUS1-RT-3′: 5′-GCCCAATTGTGCTTGCTGAA-3′ (SEQ ID NO: 14); Neo-RT-5′: 5′-TGCCTGCTTGCCGAATATC-3′ (SEQ ID NO: 15), Neo-RT-3′: 5′-ATATCACGGGTAGCCAACGC-3′ (SEQ ID NO: 16), respectively. The delta-delta CT method was used to calculate the relative mRNA expression levels in the test and control cells.

Construction of Human Antibody Libraries

The human mononuclear cells (MNCs) were purchased from Stemcell Technologies (Singapore, Cat. 70025). Total RNA was extracted by the TRIzol reagent (Invitrogen, Cat. 15596-026). 10⁸ cells were suspended in 1 ml of TRIzol and incubated at room temperature for 5 min with occasional inversion of the tube. 0.2 ml of chloroform was added and the tube was vortexed for 15 seconds and left at room temperature for 3 min before centrifuged at 12,000 g for 15 min at 4° C. The aqueous phase was transferred to a fresh 1.5 ml tube and 0.5 ml of isopropanol was added. After 10 min of incubation at room temperature, RNA was pelleted by centrifugation at 12,000 g for 10 min at 4° C. The pellet was washed with 1 ml of 75% ethanol and recollected by centrifugation at 7,500 g for 5 min at 4° C. The pellet was air-dried for 10 min at room temperature and dissolved in 30 μl of DEPC water. The tubes were left at 58° C. in a heat block for 10 min to dissolve the RNA. The mRNA was isolated from the total RNA by using Oligotex mRNA Mini Kit (Qiagen, Cat. 70022) according instructions of the manufacturer.

Additional polyA-mRNA made from human peripheral leukocytes was purchased from Clontech (Cat. 636170). The mRNAs from both sources were evaluated by PCR of 9 house-keeping gene transcripts and were confirmed to be in a good quality. They were thus combined for use in the library construction.

PCR and assembly of scFv libraries were essentially according the published protocol. The primers for making scFv libraries were listed in Table 3. First strand cDNA was generated from the mRNA mixtures of the two sources mentioned above, using SuperScript III reverse transcriptase (Life Technologies, Cat. 18080) and random hexamer primers. The genes for variable regions of heavy chain, light chain κ, and light chain λ (VH, Vκ, and Vλ) were amplified separately by PCR using GoTaq DNA polymerase (Promega, Cat. M3001). The products of this first round of PCR were then used as the templates for the second round of PCR, in which the 5′ primers of the light chain and the 3′ primers of the heavy chain shared an overlapping sequence of the linker region. In addition, a FLAG epitope tag was attached to the 3′ primers of the light chain, and a partial sequence of the signal peptide of FLO1 was included in the 5′ primers of the heavy chain. The PCR reactions for each of the variable regions were performed in a volume of 50 μl, and were first heated at 95° C. for 2 min, and then followed by 30 cycles of 95° C. for 30 sec, 57° C. for 1 min, 72° C. for 1 min and finished by a final extension at 72° C. for 5 min. In the third round of PCR, heavy and light chains were assembled and amplified with the 5′ and 3′ outer primers. The assembly PCR reactions contained equal molar mixture of the pooled heavy (VH) DNA and pooled light (Vκ, or Vλ) gene repertoires from the second round of PCR and was allowed to cycle 8 times (95° C. for 45 sec, 58° C. for 30 sec, and 72° C. for 60 sec) without primers. This was followed by the final round of PCR which was cycled 25 times (95° C. for 30 sec, 58° C. for 30 sec, and 72° C. for 60 sec) with the outer primers, and plus a final extension at 72° C. for 15 min. The final PCR reaction created a full-length scFv gene repertoire. The scFv repertoires were subjected to agarose gel electrophoresis and the DNA bands in the desired length were purified from the gel using the EZ-10 Spin Column DNA Gel Extraction Kit (Bio Basic, Singapore, Cat. B S654).

Table 3 summarizes the primers for scFv library construction.

First round PCR primers (VH):

| | |
|---|---|
| HM1 | 5′-CAGGTBCAGCTGGTGCAGTCTGG-3′ (SEQ ID NO: 17) |
| HM1/7 | 5′-CARRTSCAGCTGGTRCARTCTGG-3′ (SEQ ID NO: 18) |
| HM2 | 5′-CAGRTCACCTTGAAGGAGTCTGG-3′ (SEQ ID NO: 19) |
| HM3A | 5′-SARGTGCAGCTGGTGGAGTCTGG-3′ (SEQ ID NO: 20) |
| HM3B | 5′-GAGGTGCAGCTGKTGGAGWCYSG-3′ (SEQ ID NO: 21) |
| HM4A | 5′-CAGGTGCARCTGCAGGAGTCGGG-3′ (SEQ ID NO: 22) |
| HM4B | 5′-CAGSTGCAGCTRCAGSAGTSSGG-3′ (SEQ ID NO: 23) |
| HM5 | 5′-GARGTGCAGCTGGTGCAGTCTGG-3′ (SEQ ID NO: 24) |
| HM6 | 5′-CAGGTACAGCTGCAGCAGTCAGG-3′ (SEQ ID NO: 25) |
| HMR | 5′-AAGGGTTGGGGCGGATGCACT-3′ (SEQ ID NO: 26) |

First round PCR primers (VL kappa):

| | |
|---|---|
| LCK1A | 5′-GACATCCAGATGACCCAGTCTCC-3′ (SEQ ID NO: 27) |
| LCK1B | 5′-GMCATCCRGWTGACCCAGTCTCC-3′ (SEQ ID NO: 28) |
| LCK2 | 5′-GATRTTGTGATGACYCAGWCTCC-3′ (SEQ ID NO: 29) |
| LCK3 | 5′-GAAATWGTGWTGACRCAGTCTCC-3′ (SEQ ID NO: 30) |
| LCK4 | 5′-GACATCGTGATGACCCAGTCTCC-3′ (SEQ ID NO: 31) |
| LCK5 | 5′-GAAACGACACTCACGCAGTCTCC-3′ (SEQ ID NO: 32) |
| LCK6 | 5′-GAWRTTGTGMTGACWCAGTCTCC-3′ (SEQ ID NO: 33) |
| LCKR1 | 5′-ACACTCTCCCCTGTTGAAGCTCTT-3′ (SEQ ID NO: 34) |

First round PCR primers (VL lambda):

| | |
|---|---|
| LCL1A | 5′-CAGTCTGTGCTGACTCAGCCACC-3′ (SEQ ID NO: 35) |
| LCL1B | 5′-CAGTCTGTGBTGACGCAGCCGCC-3′ (SEQ ID NO: 36) |
| LCL2 | 5′-CARTCTGCCCTGACTCAGCCT-3′ (SEQ ID NO: 37) |
| LCL3A | 5′-TCCTATGWGCTGACWCAGCCACC-3′ (SEQ ID NO: 38) |
| LCL3B | 5′-TCTTCTGAGCTGACTCAGGACCC-3′ (SEQ ID NO: 39) |
| LCL4A | 5′-CACGTTATACTGACTCAACCGCC-3′ (SEQ ID NO: 40) |
| LCL4B | 5′-CAGCYTGTGCTGACTCAATCRYC-3′ (SEQ ID NO: 41) |
| LCL4C | 5′-CTGCCTGTGCTGACTCAGCCC-3′ (SEQ ID NO: 42) |
| LCL5 | 5′-CAGSCTGTGCTGACTCAGCC-3′ (SEQ ID NO: 43) |
| LCL6 | 5′-AATTTTATGCTGACTCAGCCCCA-3′ (SEQ ID NO: 44) |
| LCL7/8 | 5′-CAGRCTGTGGTGACYCAGGAGCC-3′ (SEQ ID NO: 45) |
| LCL9/10 | 5′-CWGCCWGKGCTGACTCAGCCMCC-3′ (SEQ ID NO: 46) |
| LCLR1 | 5′-TGAACATTCTGTAGGGGCCACTG-3′ (SEQ ID NO: 47) |
| LCLR2 | 5′-TGAACATTCCGTAGGGGCAACTG-3′ (SEQ ID NO: 48) |

Second round PCR primers (VH):

| | |
|---|---|
| FLO1SP-HM1 | 5′-GCACTAATTAATGTGGCCTCAGGACAGGTBCAGCTGGTGCAGTCTGG-3′ (SEQ ID NO: 49) |
| FLO1SP-HM1/7 | 5′-GCACTAATTAATGTGGCCTCAGGACARRTSCAGCTGGTRCARTCTGG-3′ (SEQ ID NO: 50) |
| FLO1SP-HM2 | 5′-GCACTAATTAATGTGGCCTCAGGACAGRTCACCTTGAAGGAGTCTGG-3′ (SEQ ID NO: 51) |
| FLO1SP-HM3A | 5′-GCACTAATTAATGTGGCCTCAGGASARGTGCAGCTGGTGGAGTCTGG-3′ (SEQ ID NO: 52) |
| FLO1SP-HM3B | 5′-GCACTAATTAATGTGGCCTCAGGAGAGGTGCAGCTGKTGGAGWCYSG-3′ (SEQ ID NO: 53) |
| FLO1SP-HM4A | 5′-GCACTAATTAATGTGGCCTCAGGACAGGTGCARCTGCAGGAGTCGGG-3′ (SEQ ID NO: 54) |
| FLO1SP-HM4B | 5′-GCACTAATTAATGTGGCCTCAGGACAGSTGCAGCTRCAGSAGTSSGG-3′ (SEQ ID NO: 55) |
| FLO1SP-HM5 | 5′-GCACTAATTAATGTGGCCTCAGGAGARGTGCAGCTGGTGCAGTCTGG-3′ (SEQ ID NO: 56) |
| FLO1SP-HM6 | 5′-GCACTAATTAATGTGGCCTCAGGACAGGTACAGCTGCAGCAGTCAGG-3′ (SEQ ID NO: 57) |
| LLink-HMR | 5′-GGAAGATCTAGAGGAACCACCAAGGGTTGGGGCGGATGCACT-3′ (SEQ ID NO: 58) |

Second round PCR primers (VL kappa):

| | |
|---|---|
| LLink-LCK1A | 5′-GGTGGTTCCTCTAGATCTTCCTCCTCTGGTGGCGGTGGCTCGGGCGGTGGTGGGGACATCCAGAT |
| | GACCCAGTCTCC-3′ (SEQ ID NO: 59) |
| LLink-LCK1B | 5′-GGTGGTTCCTCTAGATCTTCCTCCTCTGGTGGCGGTGGCTCGGGCGGTGGTGGGGMCATCCRGWT |
| | GACCCAGTCTCC-3′ (SEQ ID NO: 60) |
| LLink-LCK2 | 5′-GGTGGTTCCTCTAGATCTTCCTCCTCTGGTGGCGGTGGCTCGGGCGGTGGTGGGGATRTTGTGAT |
| | GACYCAGWCTCC-3′ (SEQ ID NO: 61) |
| LLink-LCK3 | 5′-GGTGGTTCCTCTAGATCTTCCTCCTCTGGTGGCGGTGGCTCGGGCGGTGGTGGGGAAATWGTGWT |
| | GACRCAGTCTCC-3′ (SEQ ID NO: 62) |
| LLink-LCK4 | 5′-GGTGGTTCCTCTAGATCTTCCTCCTCTGGTGGCGGTGGCTCGGGCGGTGGTGGGGACATCGTGAT |
| | GACCCAGTCTCC-3′ (SEQ ID NO: 63) |
| LLink-LCK5 | 5′-GGTGGTTCCTCTAGATCTTCCTCCTCTGGTGGCGGTGGCTCGGGCGGTGGTGGGGAAACGACACT |
| | CACGCAGTCTCC-3′ (SEQ ID NO: 64) |
| LLink-LCK6 | 5′-GGTGGTTCCTCTAGATCTTCCTCCTCTGGTGGCGGTGGCTCGGGCGGTGGTGGGGAWRTTGTGMT |
| | GACWCAGTCTCC-3′ (SEQ ID NO: 65) |
| FLAG-LCKR2 | 5′-CTTGTCGTCATCGTCTTTGTAGTCGAAGACAGATGGTGCAGCCACAG7-3′ |
| | (SEQ ID NO: 66) |

Second round PCR primers (VL lambda):

| | |
|---|---|
| LLink-LCL1A | 5′-GGTGGTTCCTCTAGATCTTCCTCCTCTGGTGGCGGTGGCTCGGGCGGTGGTGGGCAGTCTGTGCT |
| | GACTCAGCCACC-3′ (SEQ ID NO: 67) |
| LLink-LCL1B | 5′-GGTGGTTCCTCTAGATCTTCCTCCTCTGGTGGCGGTGGCTCGGGCGGTGGTGGGCAGTCTGTGBT |
| | GACGCAGCCGCC-3′ (SEQ ID NO: 68) |
| LLink-LCL2 | 5′-GGTGGTTCCTCTAGATCTTCCTCCTCTGGTGGCGGTGGCTCGGGCGGTGGTGGGCARTCTGCCCT |
| | GACTCAGCCT-3′ (SEQ ID NO: 69) |
| LLink-LCL3A | 5′-GGTGGTTCCTCTAGATCTTCCTCCTCTGGTGGCGGTGGCTCGGGCGGTGGTGGGTCCTATGWGCT |
| | GACWCAGCCACC-3′ (SEQ ID NO: 70) |
| LLink-LCL3B | 5′-GGTGGTTCCTCTAGATCTTCCTCCTCTGGTGGCGGTGGCTCGGGCGGTGGTGGGTCTTCTGAGCT |
| | GACTCAGGACCC-3′ (SEQ ID NO: 71) |
| LLink-LCL4A | 5′-GGTGGTTCCTCTAGATCTTCCTCCTCTGGTGGCGGTGGCTCGGGCGGTGGTGGGCACGTTATACT |
| | GACTCAACCGCC-3′ (SEQ ID NO: 72) |
| LLink-LCL4B | 5′-GGTGGTTCCTCTAGATCTTCCTCCTCTGGTGGCGGTGGCTCGGGCGGTGGTGGGCAGCYTGTGCT |
| | GACTCAATCRYC-3′ (SEQ ID NO: 73) |
| LLink-LCL4C | 5′-GGTGGTTCCTCTAGATCTTCCTCCTCTGGTGGCGGTGGCTCGGGCGGTGGTGGGCTGCCTGTGCT |
| | GACTCAGCCC-3′ (SEQ ID NO: 74) |
| LLink-LCL5 | 5′-GGTGGTTCCTCTAGATCTTCCTCCTCTGGTGGCGGTGGCTCGGGCGGTGGTGGGCAGSCTGTGCT |
| | GACTCAGCC-3′ (SEQ ID NO: 75) |
| LLink-LCL6 | 5′-GGTGGTTCCTCTAGATCTTCCTCCTCTGGTGGCGGTGGCTCGGGCGGTGGTGGGAATTTTATGCT |
| | GACTCAGCCCCA-3′ (SEQ ID NO: 76) |
| LLink-LCL7/8 | 5′-GGTGGTTCCTCTAGATCTTCCTCCTCTGGTGGCGGTGGCTCGGGCGGTGGTGGGCAGRCTGTGGT |
| | GACYCAGGAGCC-3′ (SEQ ID NO: 77) |
| LLink- | 5′-GGTGGTTCCTCTAGATCTTCCTCCTCTGGTGGCGGTGGCTCGGGCGGTGGTGGGCWGCCWGKGCT |
| LCL9/10 | GACTCAGCCMCC-3′ (SEQ ID NO: 78) |
| FLAG-LCLR3 | 5′-CTTGTCGTCATCGTCTTTGTAGTCAGAGGASGGYGGGAACAGAGTGAC-3′ |
| | (SEQ ID NO: 79) |

Outer primers:

| | |
|---|---|
| 5′outer | 5′-ATCTCAGCGGCCGCACTAATTAATGTGGCCTCAGGA-3′ (SEQ ID NO: 80) |
| NotI | |
| 3′outer | 5′-CGCAGTAAGCTTGTCGTCATCGTCTTTGTAGTC-3′ (SEQ ID NO: 81) |
| HindIII | |

To clone the full-length scFv DNA into pRS315, the final scFv PCR products and pRS315 were each digested with NotI and HindIII at 37° C. for overnight. The digested scFv DNA and vector were then purified with EZ-10 Spin Column as mentioned above, and mixed together in a ligation reaction at a vector/insert molar ratio of 1:3, and left at room temperature for overnight with the T4 DNA ligase (NEB, Cat. M0202). The ligated DNA was transduced into the E. coli Top10 cells by electroporation using a MicroPulser Electroporator (Bio-Rad). The cells were then transferred into 2×YT broth and incubated with shaking for 1 hr at 37° C. 20 μl of the culture was taken for serial dilutions and plating on 2×YT plates for colony counting. The rest of the culture was allowed to incubate for additional 2 hrs. The cells were then collected and resuspended in 2×YT, aliquoted, and stored at −80° C. as glycerol stocks. Aliquots of the cell were taken for DNA preparation by Gene JET Plasmid Maxiprep Kit (Thermo Fisher Scientific, Cat. K0491) when needed.

To generate a pre-made antibody library in the yeast vector pKT131, the final PCR reaction that created a full-length scFv gene repertoire described above was performed with outer primers containing NotI and AscI sites, respectively. The PCR product was digested overnight at 37° C. with NotI and AscI, and ligated with the pKT131 vector similarly digested with the restriction enzymes at a vector/PCR product ratio of 1:3. The ligated DNA was then introduced into E. coli Top10 cells by electroporation. 5×10⁹ independent clones were obtained.

Antibody Library Screening

Transformation by Gap-Repair:

The scFv library made in pRS315 was amplified by PCR using the DreamTaq DNA Polymerase (Thermo Fisher Scientific). The following mixtures were prepared in a 50 μl of reaction on ice: 5 μl of 10× DreamTaq Buffer, 1 μl of dNTP Mix (final 200 μM), 2.5 μl of gap repair forward primer (5′ATGTTTTTGGCAGTCTTTACACTTCTGGCACTAATTAATGTGGCCTCAGGA3′ (SEQ ID NO: 82), final 0.5 μM), 2.5 μl of gap repair reverse primer (5′GATAAGATTTAAATATAAAAGATATGCAACTAGAAAAGTCTTATCAATCTCCG GATCCTCATTACTTGTCGTCATCGTCTTTGTAGTC3′ (SEQ ID NO: 83), final 0.5 μM), 10 μl (100 ng) of pRS315-scFv library DNA, 0.25 μl (1.25 U) of DreamTaq DNA Polymerase, and 28.75 μl of H₂O. The mixtures were first heated at 95° C. for 2 min, followed by 5 cycles of 95° C. for 30 sec, 58° C. for 30 sec, 72° C. for 1 min and followed by 25 cycles of 95° C. for 30 sec, 65° C. for 30 sec, 72° C. for 65 sec, and concluded with a final extension at 72° C. for 6 min. The amplified scFv (˜900 bp) was purified by agarose gel electrophoresis as mentioned above.

To prepare the host cell for transformation, the overnight cultures of a yeast strain (YKT099, for example) in YPD at 28° C. was diluted into 50 ml of fresh YPD at the density of OD₆₀₀=0.3, and was allowed to grow for another 5-6 hrs at 28° C., till OD₆₀₀ reached 1.3-1.6. The cells were collected by centrifugation at 3,000 g for 4 min at 4° C. and washed twice with 50 ml of ice-cold water and once with ice-cold electroporation buffer (1 M Sorbitol, 1 mM CaCl₂). The cells were then pelleted and resuspended in 20 ml of 0.1 M LiAc, 10 mM DTT, and incubated at 28° C. with shaking for 30 min. The cells were collected by centrifugation at 3,000 g for 4 min and washed once with the ice-cold electroporation buffer, resuspended in 0.5 ml of the ice-cold electroporation buffer, and kept on ice till use.

To 400 μl of the yeast competent cells add 4 μg of the NotI-SacII digested pKT103 DNA and 12 μg of the PCR-amplified scFv DNA, and transfer the mixture to a pre-chilled 0.2 cm cuvette. Keep the cuvette on ice for 5 minutes before the electroporation, which was done at 2.5 kV and 25 μF with a MicroPulser Electroporator (Bio-Rad). Transfer the cells to 8 ml of 1:1 mix of 1 M sorbitol/YPD media and incubate the cells at 28° C. for 1 hr with mild shaking. The cells were pelleted at 3,000 g for 5 min and resuspended in 20 ml of 2×YPD media. Plate 10-fold serial dilutions on Leu-dropout plates for transformation efficiency measurement. The rest of the cells were pelleted and resuspended in 2 ml of leucine dropout medium (SC-Leu) and 200 μl of the suspension were taken to spread on each SC-Leu plates supplemented with either 200-400 μg/ml of G418 to be incubated at 30° C., or 20-40 μg/ml of G418 to be incubated at 37° C.

Transformation of Pre-Made Libraries:

Inoculate an aliquot of the overnight culture into 50 ml of YPD media at OD₆₀₀=0.4. Grow the cells for 4.5-5 hours. The cells are collected by centrifugation at 3000 g for 3 min and washed twice with ice-cold water and once with ice-cold 1 M Sorbitol. Re-suspend the cell pellet in 20 ml of 0.1 M LiAc/10 mM DTT and incubate at 30° C. for 30 min. Cells are pelleted and washed once with ice-cold 1 M Sorbitol, and re-suspended in 1 M Sorbitol to a final volume of 400 μl.

Add 10 μg of library DNA for each 400 μl of cells. Mix gently and transfer the mixture to a pre-chilled BioRad GenePulser cuvette (0.2 cm). Leave the cuvette on ice for 5 min until electroporation. Electroporate the cells at 1.5 kV (typically for 3.5 to 4.5 milliseconds). Transfer the cells immediately from the cuvette to 20 ml of YPD containing 0.5 M sorbitol. Incubate the cells at 30° C. for 1 hour before plating on selective media.

### Example 1

Brief Overview of the Platform

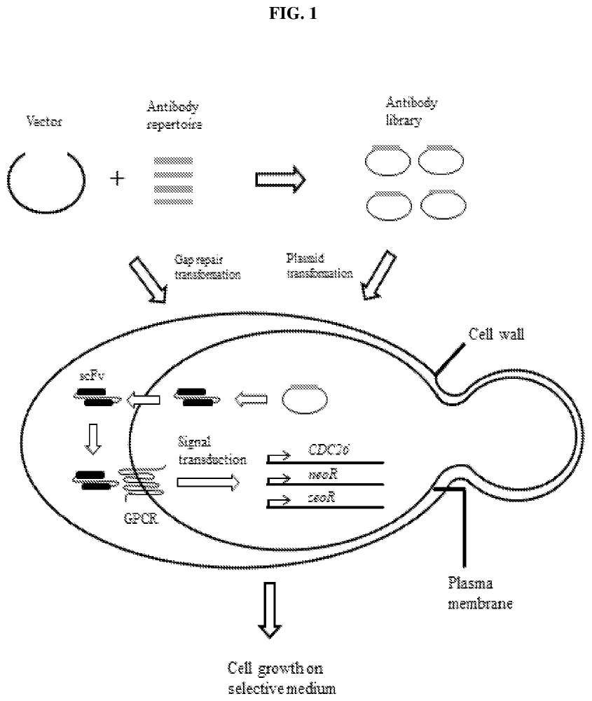

The platform illustrated in FIG. 1 is a typical phenotypic screen based on autocrine signaling. The agonist antibody from a combinatorial antibody library, which is introduced into the cell by high efficiency transformation, is produced and secreted into the periplasmic space between cell wall and the plasma membrane where it or a significant portion of it is retained due to its size. When the antibody finds its epitope on the receptor (Ste2p or a human GPCR receptor), it binds to the receptor and activates it, and triggers a cascade of kinase reactions which activate the Ste12p transcription factor at the end of the kinase cascade. The activated Ste12p then binds to the promoter region of a group of pheromone-inducible genes and stimulates their transcription. The promoters of two of such genes, FUS2 and FIG1, were chosen to drive the expression of reporter genes, CDC26 and the antibiotic resistance genes (the neomycin resistance gene, neoR, and the Zeocin resistance gene, zeoR), resulting in cell growth in the presence of the antibiotics, or at 37° C., or both.

### Example 2

Construction of Library, Vectors and Host Strains

The scFv repertoire was first made in a common shuttle vector pRS315 at the diversity of >10¹⁰, which is comparable to, or better than, most of the phage display antibody libraries. Sequence analysis of randomly chosen library clones indicated that >80% of the library members contained antibody sequences that were correctly assembled into the vector. When a screen was performed, the repertoire was introduced into the screening cells by PCR and gap-repair with the screening vector pKT103. In the process, the scFv fragments were joined to the FLO1 secretion signal sequence and placed under the TEF1 promoter.

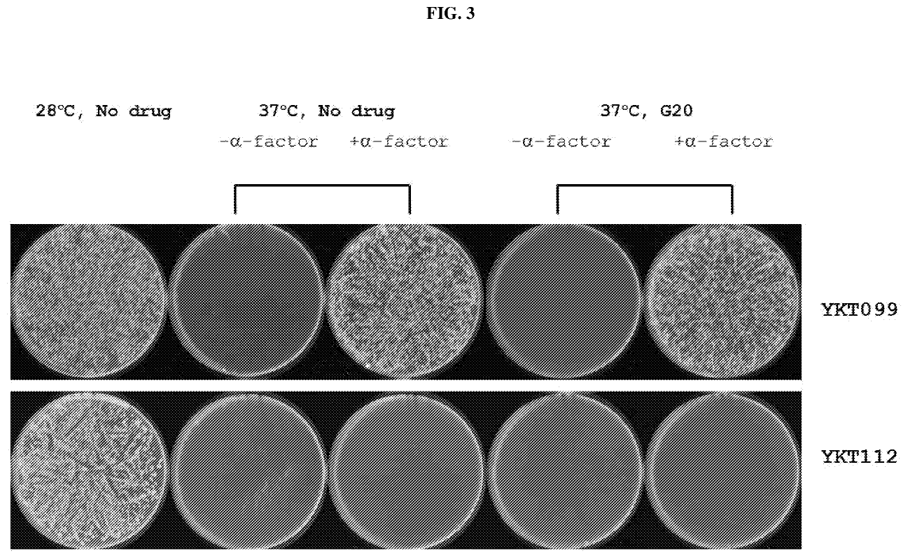

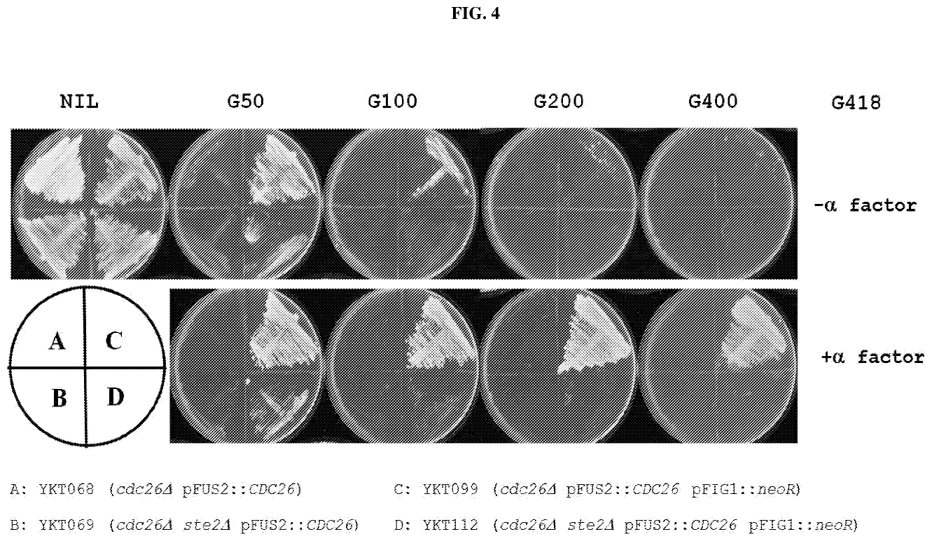

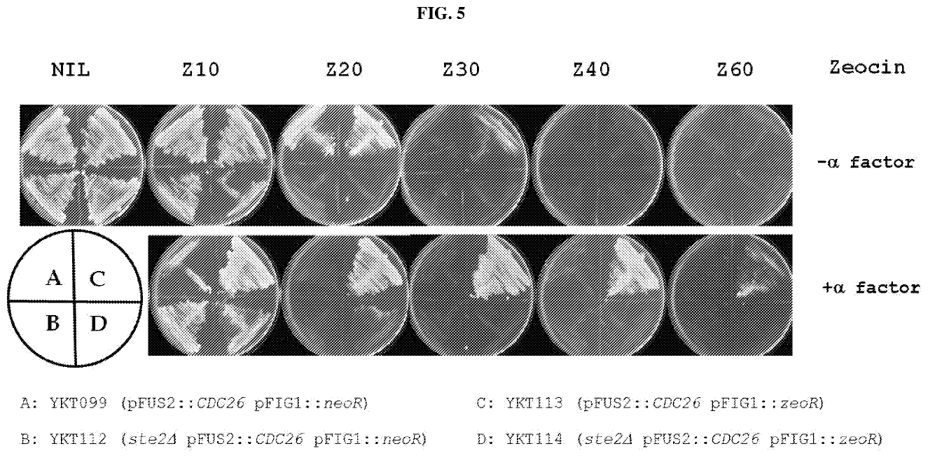

To construct the strains suitable for the purpose of this platform, some modifications to the Ste2p signaling pathway have to be made. First, like in other experiments in which the yeast pheromone signaling pathway was adopted for studies of heterologous GPCRs, two gene deletions, far1Δ and sst2Δ, were necessary for perpetuating cell division under the situation of pathway activation and for enhancing the sensitivity of the signal detection, respectively. The multi-functional and non-essential protein Far1p is required for α-factor induced cell cycle arrest, which is but one of many consequences of the pathway stimulation. Loss of the SST2 function increases the receptor/G protein coupling and therefore attenuates the down-regulation of the pathway. For sensitive and easy signal reporting, this study used antibiotic resistance genes, neoR or zeoR, and a reporter system conferring temperature sensitivity, cdc26, instead of the commonly followed method of histidine auxotroph. CDC26 encodes a subunit of the anaphase-promoting complex (APC), which is involved in the regulation of metaphase/anaphase transition. Loss of the CDC26 function is lethal at high temperature (37° C.). Cells (YKT068 and YKT099) carrying the cdc26 deletion and wild type CDC26 under the control of the FUS2 promoter were temperature sensitive for growth at 37° C. in the absence of α-factor, but grew normally in the presence of the pheromone (FIG. 3). Similarly, cells (YKT099 or YKT113) containing neoR or zeoR genes under the FIG1 promoter were able to grow in the media containing antibiotics G418 or Zeocin, respectively, only in the presence of α-factor (FIGS. 4 and 5). In all these cases, the α-factor-supported cell growth was abolished when the receptor gene STE2 was deleted. These data demonstrate that these strains were adequate for use in the screening scheme that was envisaged (FIG. 1). By employing multiple and yet convenient selection criteria, this study demonstrated the potential of this screening platform for discovery of agonistic antibodies of GPCRs highly efficient and robust.

It should be pointed out that any drug resistance genes and any mutations conferring temperature sensitivity can be used instead of neoR, zeoR, and cdc26, as long as their backgrounds are at low or manageable levels.

### Example 3

Agonistic Antibodies Against Ste2p

The platform was firstly used to search for antibodies that were able to act as agonists for Ste2p, the yeast GPCR receptor. YKT099 was transformed with the scFv library and the transformed cells were spread on SC-Leu plates (to select for the plasmid) which also contained G418 at 20-40 μg/ml or 300-400 μg/ml, and were incubated at 37° C. or 30° C., respectively. The “high temperature/low drug” selection condition was biased towards the CDC26 expression and the “low temperature/high drug” condition interrogated mainly the neoR expression. The colonies that grew up on the plates under each selection regimen were collected and pooled together with each pool containing 10 or so individual colonies. Plasmids were extracted from pooled cells and used again to transform YKT099. This time, the transformants were screened with a reversed selection protocol. For example, if the first round of screening used the high temperature/low drug selection, the second round will use low temperature/high drug selection, and vice versa. This enrichment procedure was repeated once again. Typically, after three rounds of enrichment, nearly all the transformants became positive for growth on the selection plate. The colonies were collected for the last time and their plasmid DNA was extracted and analyzed.

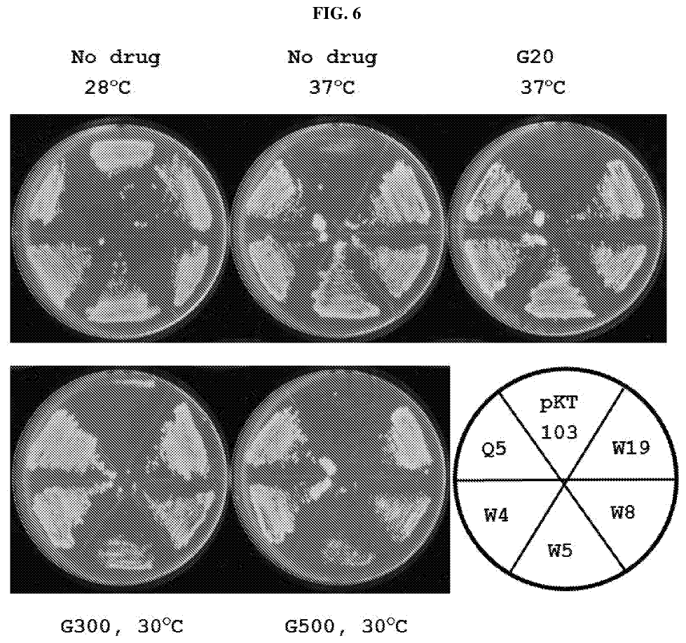

From total of 5×10⁸ transformants, a dozen or so plasmids were identified that repeatedly produced colonies on the selection plates. FIG. 6 showed five of these isolates that displayed variable degrees of activities when interrogated with different selection conditions. W5 and W8, for example, appeared to support the cell growth at 37° C. as efficiently as the other three isolates but were evidently less so on the plates that contained high concentrations of G418 at 30° C. (FIG. 6). One of the isolates that displayed stronger activity, Q5, was chosen for further characterization.

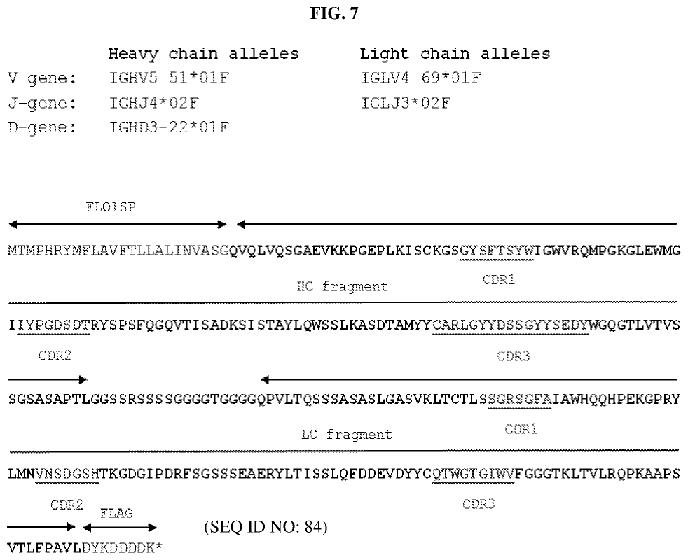

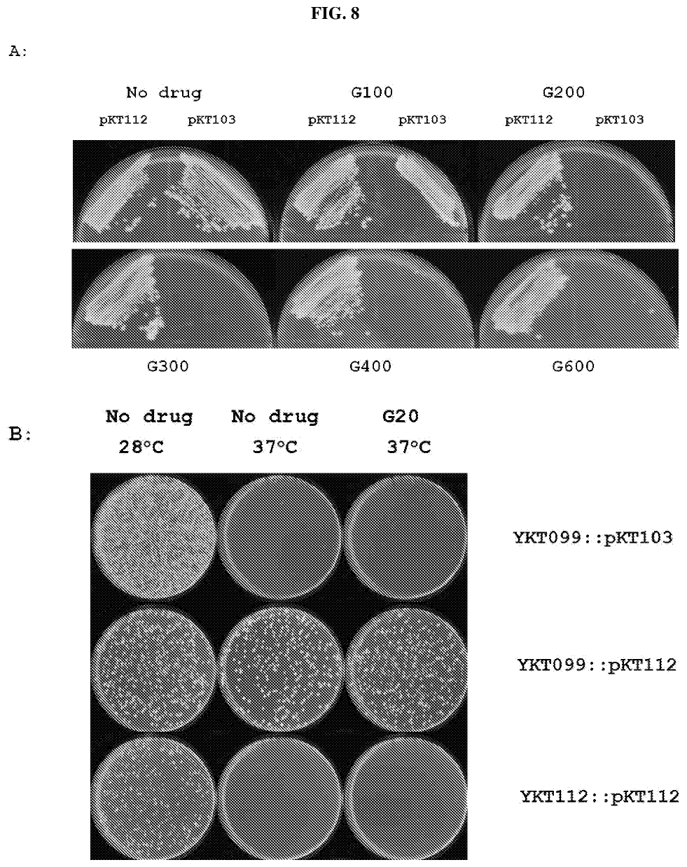

The Q5 gene encodes a 308 amino acid protein, including the N-terminal Flo1 signal peptide and the C-terminal FLAG epitope tag. The amino acid sequence of Q5 was given in FIG. 7. The plasmid expressing Q5, pKT112, demonstrated a potent activity in supporting the survival of the YKT099 cells under the selection conditions. YKT099 cells hosting pKT112 were able to grow healthily on the G418-containing plates at all concentrations tested, up to 600 μg/ml (FIG. 8A). Similarly, pKT112 enabled YKT099 cells to grow normally at 37° C. (FIG. 8B). As the CDC26 and neoR genes were expressed by two different pheromone-inducible promoters in YKT099 (pFUS2 and pFIG1), it is likely that the cell survival phenotype was resulted from the activation of the Ste12p-initiated transcription, rather than from some physiological changes in the cells brought about by Q5 that were unrelated to the pheromone signaling pathway but enhanced the resistance to the lethal effects of the selection conditions by other means such as increased thermal tolerance or decreased membrane permeability for the antibiotic. In support of this conception, the YKT112 strain, which differs from YKT099 in lacking the pheromone receptor Ste2p, could not be rescued from the lethality of high temperature treatment by Q5 under the same conditions (FIG. 8B), indicating that the function of Q5 was mediated through the Ste2p signaling pathway.

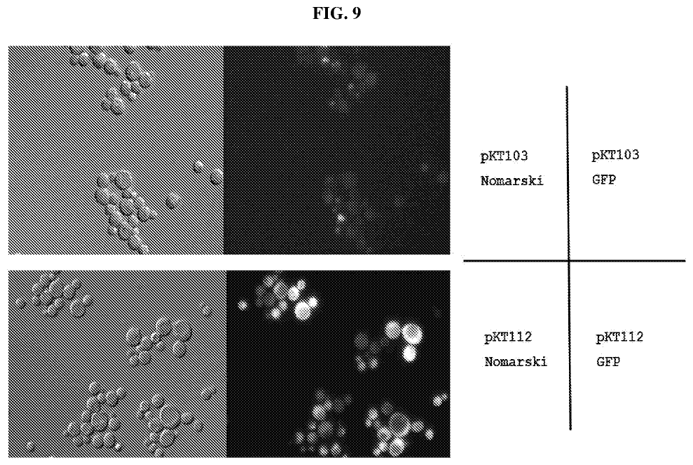

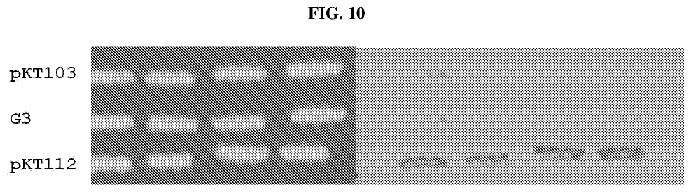

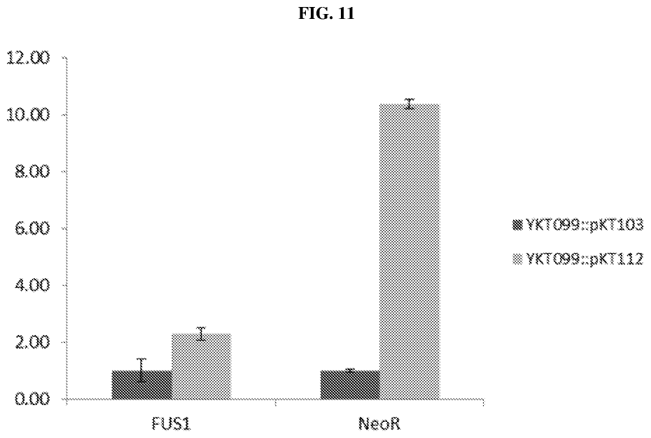

To further demonstrate Q5's function in promoting expression of pheromone-inducible genes, the expression of two other reporter genes were examined: GFP and LacZ in YKT099 and YKT109, respectively, both of which were driven by the FIG1 promoter, and found both genes to be markedly stimulated by the Q5-containing plasmid pKT112 (FIGS. 9 and 10). When the transcripts of the neoR and FUS1 genes were directly analyzed by real-time quantitative PCR, it was revealed that they were elevated 10 fold and 2 fold in YKT099, respectively, by Q5 (FIG. 11).

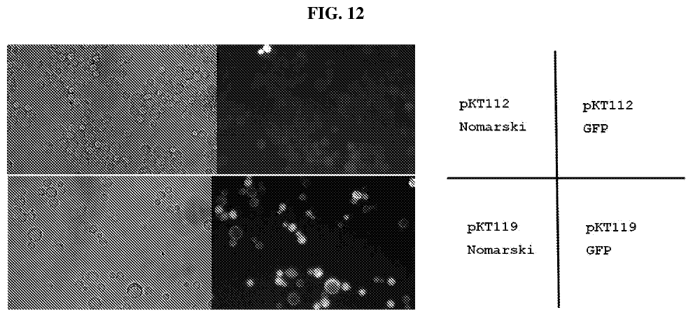

To examine the cellular localization of Q5, the GFP coding sequence was cloned into pKT112 in-frame with, and C-terminal to, Q5. The plasmid so created, pKT119, was introduced into BJ5465 cells and it was found that about 30-50% of the cells clearly showed a cell surface localization of GFP (FIG. 12), whereas the control cells which contained Q5 devoid of GFP (pKT112) showed no GFP signals (FIG. 12). This cellular localization pattern of Q5 is within the expectation and is consistent with its role as an agonist of the Ste2p receptor.

In conclusion, the human antibody fragment Q5 was isolated by the autocrine screening platform as an agonist for the yeast GPCR receptor Ste2p, on the basis of its activity to stimulate the expression of the pheromone-inducible genes and do so in a Ste2p-dependent manner. These data demonstrate that the yeast autocrine screening method is effective and convenient for identifying functional antibodies against GPCR receptors.

### Example 4

The Activity of Q5 was Attenuated by a GPI Anchor

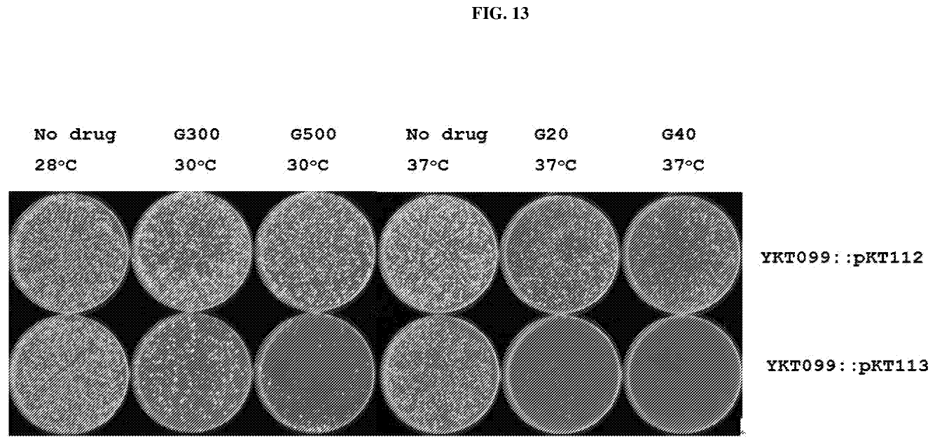

Previously, Ishii et al. demonstrated an autocrine signaling system in yeast using the human GPCR SSTR5 and its ligand, somatostatin-14, which was anchored on the cell surface by a 41 amino acid glycosylphosphatidylinositol (GPI) attachment domain from the yeast cell wall protein Flo1p (Flo42). The same GPI anchor element (Flo42 with a flexible linker) was used to construct the vector pKT047, which was tested in an antibody library screening in the same way as with pKT103. The positive clones obtained from pKT047-based screening were fewer in number and weaker in activity, compared with the ones yielded from the pKT103-based screening. To directly compare the activity of the anchored and the unanchored agonist antibody, Q5 was transferred from pKT112 to pKT047 to fuse with the Flo42 anchor at the C-terminus of Q5 (pKT113). As shown in FIG. 13, the activity of Q5 in supporting the growth of YKT099 cells under the selection conditions was greatly weakened by GPI attachment (FIG. 13). This result indicates that the free movement of Q5 in the periplasmic space is important for its interaction and activation of Ste2p. As the Q5 protein has an apparent molecular weight of 30 kD, it may not require an anchor to have a significant amount of its protein product to be retained in the periplasmic space. However, when the similar screen is to be performed with smaller protein constructs such as a single domain antibody fragment library or an oligo-peptide library, it may be necessary to use membrane or cell wall anchors (e.g., a transmembrane motif or GPI anchor element) to confine these molecules to the periplasmic space, as the yeast cell wall may be freely permeable to them.

In summary, a yeast phenotypic screening system was constructed based on autocrine signaling for identification of human antibody molecules that act as agonists of GPCR receptors. The effectiveness of this platform was demonstrated by isolating antibody fragments that can activate the yeast Ste2p-mediated pheromone signaling pathway. Compared with other antibody discovery platforms, the platform described in this study is more convenient, efficient, and uncostly for isolation of functional antibodies targeted at the GPCR proteins. Conceivably, this yeast autocrine screening system can also be used for isolation of antagonistic antibodies, if operated conversely.

### EQUIVALENTS

The foregoing examples are presented for the purpose of illustrating the invention and should not be construed as imposing any limitation on the scope of the invention. It will readily be apparent that numerous modifications and alterations may be made to the specific embodiments of the invention described above and illustrated in the examples without departing from the principles underlying the invention. All such modifications and alterations are intended to be embraced by this application.

1-29. (canceled)

30. A method for identifying a functional antibody or antigen-binding protein or a fragment thereof that is capable of binding to, and stimulating the activity of, a target transmembrane protein comprising:
a) providing yeast cells transformed with yeast expression vectors encoding a library of antibodies or antigen-binding proteins or fragments thereof, wherein said yeast cells express said target transmembrane protein;
b) expressing said library of antibodies or antigen-binding proteins or fragments thereof in the yeast cells, wherein said expressed antibodies or antigen-binding proteins or fragments thereof are secreted into the periplasmic space of said yeast cells;
c) incubating the yeast cells in or on a selective medium or under a restrictive temperature, or a combination thereof; and
d) detecting a predetermined phenotype in the yeast cells, wherein manifestation of the predetermined phenotype is indicative of binding of the functional antibody or antigen-binding protein or a fragment thereof to said target transmembrane protein and stimulation of the activity of said target transmembrane protein by the functional antibody or antigen-binding protein or a fragment thereof.

31. The method of claim 30, wherein the target transmembrane protein is a G-protein coupled receptor, or GPCR.

32. The method of claim 30, wherein the functional antibody or antigen-binding protein or a fragment thereof is an agonist of GPCR.

33. The method of claim 30, wherein the library of antibodies or antigen-binding proteins or fragments thereof is a single-chain variable fragment (scFv), a single domain (VHH), a Fab, a polypeptide or an oligo-peptide library.

34. The method of claim 30, wherein the yeast cells in step a) comprise one or more reporter genes, wherein expression of the one or more reporter genes is inducible by activation of a GPCR-mediated signal transduction pathway; optionally wherein the GPCR-mediated signal transduction pathway is based on the yeast mating pheromone signaling pathway; optionally wherein the one or more reporter genes is an antibiotic resistance gene, a temperature-sensitivity complementing gene, or a fluorescent protein gene, or combinations thereof; and optionally wherein the antibiotic resistance gene is the G418-resistance gene or the zeocin-resistance gene, or both.

35. The method of claim 34, wherein the fluorescent protein gene is the green fluorescent protein gene GFP or the red fluorescent protein gene RFP or the yellow fluorescent protein gene YFP or the blue fluorescent protein gene BFP, or the cyan fluorescent protein gene CFP.

36. The method of claim 30, wherein the predetermined phenotype is cell growth in the presence of one or more antibiotics, or cell growth at a restrictive temperature, or generation of fluorescence due to expression of a reporter fluorescent protein gene, or combinations thereof.

37. The method of claim 30, wherein the target transmembrane protein is expressed on the plasma membrane of the yeast cells.

38. The method of claim 33, wherein the polypeptide or oligo-peptide library is a random or partially random peptide library that is not derived from antibody sequences; optionally wherein the oligo-peptide library comprises peptides longer than 5 amino acids.

39. The method of claim 30, wherein the yeast is Saccharomyces cerevisiae or Schizosaccharomyces pombe.

40. The method of claim 30, wherein a nucleic acid sequence encoding the antibodies or antigen-binding proteins or fragments thereof is operably linked to a yeast promoter; optionally wherein the nucleic acid sequence encoding the antibodies or antigen-binding proteins or fragments thereof further comprises a secretion signal sequence, which facilitates the localization of the said antibodies or antigen-binding proteins or fragments thereof to the periplasmic space of the yeast cells; and optionally wherein the secretion signal sequence is encoded by the FLO1 gene or the yeast mating pheromone gene MFα1.

41. The method of claim 30, wherein the antibodies or antigen-binding proteins or fragments thereof are coupled to an anchor element for localization in the periplasmic space of yeast cells; optionally wherein the anchor element is a glycosylphosphatidylinositol (GPI) attachment protein or a transmembrane domain.

42. The method of claim 30, wherein the selective medium comprises one or more antibiotics; optionally wherein the selective medium is used at a temperature range of 30 to 40° C.

43. The method of claim 30, wherein the functional antibody or antigen-binding protein or a fragment thereof is a monoclonal, recombinant, polyclonal, chimeric, humanised, bispecific and heteroconjugate antibodies, a single variable domain, a domain antibody, antigen-binding fragments, immunologically effective fragments, single-chain variable fragments, a single chain antibody, a univalent antibody lacking a hinge region, a minibody, a diabody, a polypeptide or oligo-peptide comprising an amino acid sequence not derived from an antibody.
